(19) United States
(12) Patent Application Publication
(10) Pub. No.: US 2014/0038248 A1
(43) Pub. Date: Feb. 6, 2014

(54) PRODUCT OF FATTY ACID ESTERS FROM BIOMASS POLYMERS

(71) Applicant: The Regents of the University of California, Oakland, CA (US)

(72) Inventors: Jay D. KEASLING, Berkeley, CA (US); Yisheng (Connie) KANG, Albany, CA (US); Eric J. STEEN, Berkeley, CA (US); Gregory BOKINSKY, Berkeley, CA (US)

(73) Assignee: THE REGENTS OF THE UNIVERSITY OF CALIFORNIA, Oakland, CA (US)

(21) Appl. No.: 13/960,435

(22) Filed: Aug. 6, 2013

Related U.S. Application Data
(63) Continuation of application No. 13/318,474, filed on Jan. 17, 2012, now abandoned.
(60) Provisional application No. 61/174,960, filed on May 1, 2009.

(51) Int. Cl. C12P 7/64

(52) U.S. Cl. CPC C12P 7/649
USPC 435/134; 435/252.33

(57) ABSTRACT

The invention provides consolidated bioprocessing methods and host cells. The host cells are capable of directly converting biomass polymers or sunlight into biodiesel equivalents and other fatty acid derivatives. In particular, the invention provides a method for producing biodiesel equivalents and other fatty acid derivatives from a biomass polymer including providing a genetically engineered host cell, culturing the host cell in a medium containing a carbon source such that recombinant nucleic acids in the cell are expressed, and extracting biodiesel equivalents and other fatty acid derivatives from the culture.

### CROSS-REFERENCE TO RELATED APPLICATIONS

This application is a continuation of U.S. patent application Ser. No. 13/318,474, filed Nov. 1, 2011, which is a U.S. National Phase patent application of PCT/US2010/033299, filed Apr. 30, 2010, which claims the benefit of U.S. Provisional Patent Application No. 61/174,960, filed May 1, 2009, all of which are hereby incorporated by reference in the present disclosure in their entireties.

### FIELD OF THE INVENTION

The present disclosure relates to methods and compositions for the production of fatty acid esters and other fatty acid derivatives from cellulosic biomass.

### BACKGROUND OF THE INVENTION

Fatty acid biosynthesis is central to production of many medically and industrially important compounds including omega-3 fatty acids (EPA & DHA), oils, biodiesel, fatty alcohols and waxes. Biodiesel is a superior fuel to gasoline, ethanol, and the “higher” chain alcohols (including butanol) that have been thus far produced because it is non-toxic, immiscible with water, energy dense, and lacks the major pollutants of petroleum-derived diesel (SOx, NOx, heavy metals, and aromatics). However, significant environmental and economic costs are associated with growing vegetable oil crops, extracting the oils and modifying them for use as fuel (Hill et al. 2006). Thus, there has been significant interest in producing oils from microorganisms, and much research has been focused on those organisms that accumulate significant amounts of lipid. Currently, production of oils from these organisms requires costly extraction methods, and the need for the production of oils from microorganisms that can produce lipids but do not require costly extraction methods still remains.

Although E. coli lacks an inherent capacity for fat accumulation (in contrast to lipid-accumulating microbes such as oleaginous yeasts, algaes, etc), it is already known to secrete fatty acids at low levels, a characteristic that eliminates product extraction costs commonly associated with the production of ethanol, butanol, higher-chain alcohols, and biodiesel from lipid-accumulating crops or microbes (Hall and Ratledge 1977; Brown 1969; Knights et al. 1970; Jiang and Cronan 1994). Utilizing E. coli to produce these molecules provides advantages over the traditional methods of fatty acid-based biofuel production, which relies upon harsh conditions for chemical catalysis and produces wasteful byproducts like glycerol. However, previous demonstrations of E. coli's capacity to produce biofuels have fallen short of the production levels required to be industrially relevant because of product toxicity and low titer (Atsumi et al. 2008; Fischer et al. 2008).

It has been shown that E. coli can produce fatty acid ethyl esters (FAEEs), a biodiesel equivalent, by esterifying exogenously-added fatty acids with endogenously-produced ethanol (Kalscheuer et al. 2006). However, this process would not be economically viable due to the high cost of fatty acids. Furthermore, other groups have demonstrated the production of FAEEs by modifying the native E. coli fatty acid biosynthesis and β-oxidation pathways with exogenously added ethanol to the growth media (WO 2007/136762, WO 2008/119082, WO 2009/009391). However, the need remains to engineer an E. coli cell capable of producing biodiesel equivalent without the addition of exogenous substrates.

Although production of second-generation biofuels like FAEEs from sugar has many advantages over ethanol production from sugar, sourcing that sugar from the large available biomass reserves offers an even greater advancement. Unfortunately, sourcing sugar from cellulosic biomass requires the use of costly enzymes to liberate sugars from pretreated cellulose and hemicellulose.

Thus, a further need exists for a consolidated bioprocess in which cells produce biodiesel equivalents and other fatty-acid derived chemicals directly from an input of cellulosic biomass without the addition of exogenous substrates or enzymes.

### BRIEF SUMMARY OF THE INVENTION

Described herein are consolidated bioprocessing methods and host cells. The host cells are capable of producing biodiesel equivalents and other fatty acid derivatives. In certain embodiments, the host cells have the ability to degrade plant biomass and utilize it as a sole carbon source for production of a biodiesel equivalent and other fatty acid derivatives.

Thus, one aspect includes a method for producing fatty acid ethyl esters from a carbon source, by providing a host cell, wherein the host cell includes one or more recombinant nucleic acids encoding a thioesterase, a fatty acyl-coA synthetase, an acyl-transferase, an alcohol dehydrogenase, and a pyruvate decarboxylase, culturing the host cell in a medium to form a culture such that the one or more recombinant nucleic acids are expressed in the cell, wherein the medium includes a carbon source for the host cell, and extracting fatty acid ethyl esters from the culture.

Another aspect further includes a method for producing fatty acid ethyl esters from a biomass polymer, by first providing a host cell, wherein the host cell includes one or more recombinant nucleic acids encoding a thioesterase, a fatty acyl-coA synthetase, an acyl-transferase, an alcohol dehydrogenase, a pyruvate decarboxylase, and one or more biomass polymer-degrading enzymes, wherein the one or more biomass polymer-degrading enzymes are secreted from the host cell, next culturing the host cell in a medium to form a culture such that the one or more recombinant nucleic acids are expressed in the cell, wherein the medium contains a biomass polymer as a carbon source for the host cell, and then extracting fatty acid ethyl esters from the culture. In certain embodiments the host cell contains an endogenous nucleic acid encoding a fatty acyl-coA dehydrogenase. In certain embodiments, the host cell is modified such that expression of the fatty acyl-coA dehydrogenase is attenuated relative to the level of expression in a non-modified cell. In other embodiments, the host cell is a bacterial cell, a fungal cell, a cyanobacterial cell, a plant, animal, or human cell. In further embodiments, the host cell is an E. coli cell or a yeast cell. In certain embodiments, one or more of the following is true, the thioesterase is ltesA from E. coli, the fatty acyl-coA synthetase is fadD from E. coli, the alcohol dehydrogenase is adhB from Zymomonas mobilis, the pyruvate decarboxylase is pdc from Zymomonas mobilis, or the acyl-transferase is the wax ester synthase atfA from Acinetobacter strain ADP1. In certain embodiments, the biomass polymer is hemicellulose. In certain embodiments, the hemicellulose is xylan. In certain embodiments, the one or more biomass polymer-degrading enzymes are a xylanase and a protein containing an endoxylanase catalytic domain. In a further embodiment, the xylanase is xsa from Bacteroides ovatus or Gly43F from Cellvibrio japonicus. In another embodiment, the endoxylanase catalytic domain is from xyn10B from Clostridium stercorarium. In certain embodiments, the biomass polymer is cellulose. In further embodiments, the one or more biomass polymer-degrading enzymes are a protein containing a cellobiohydrolase catalytic domain, a beta-glucosidase, and a protein containing a cellulase catalytic domain. In further embodiments, one or more of the following is true, the cellobiohydrolase catalytic domain is from cel6A from Cellvibrio japonicus, the beta-glucosidase is cel3B from Cellvibrio japonicus, or the cellulase catalytic domain is from cel from Bacillus sp. D04. In certain embodiments, the culturing medium does not comprise free fatty acids or alcohol. In certain embodiments, the biomass polymer is mannan. In further embodiments, the one or more biomass polymer-degrading enzymes are an endomannanase, an exomannanase, and an alpha-galactosidase. In further embodiments, one or more of the following is true, the endomannanase is Man26A from Cellvibrio japonicus, the exomannanase is Man5D from Cellvibrio japonicus, or the alpha-galactosidase is Aga27A from Cellvibrio japonicus.

Another aspect of the invention includes a genetically modified host cell, containing one or more recombinant nucleic acids encoding a thioesterase, a fatty acyl-coA synthetase, an acyl-transferase, an alcohol dehydrogenase, and a pyruvate decarboxylase,

Another aspect of the invention includes a genetically modified host cell, containing one or more recombinant nucleic acids encoding a thioesterase, a fatty acyl-coA synthetase, an acyl-transferase, an alcohol dehydrogenase, a pyruvate decarboxylase, and one or more biomass polymer-degrading enzymes, wherein the one or more biomass polymer-degrading enzymes are secretory enzymes. In certain embodiments, the host cell contains an endogenous nucleic acid encoding a fatty acyl-coA dehydrogenase. In further embodiments, the host cell is modified such that expression of the fatty acyl-coA dehydrogenase is attenuated relative to the level of expression in a non-modified cell. In other embodiments, the host cell is a bacterial cell, a fungal cell, a cyanobacterial cell, a plant, animal, or human cell. In further embodiments, the host cell is an E. coli cell or a yeast cell. In certain embodiments, one or more of the following is true, the thioesterase is ltesA from E. coli, the fatty acyl-coA synthetase is fadD from E. coli, the alcohol dehydrogenase is adhB from Zymomonas mobilis, the pyruvate decarboxylase is pdc from Zymomonas mobilis, or the acyl-transferase is the wax ester synthase atfA from Acinetobacter strain ADP1. In certain embodiments, the host cell also contains recombinant nucleic acid encoding a naturally secreted protein, wherein the secreted protein is fused to the one or more biomass polymer-degrading enzymes. In further embodiments, the naturally secreted protein is OsmY from E. coli. In certain embodiments, the one or more biomass polymer-degrading enzymes are a xylanase and a protein containing an endoxylanase catalytic domain. In a further embodiment, the xylanase is xsa from Bacteroides ovatus or Gly43F from Cellvibrio japonicus. In another embodiment, the endoxylanase catalytic domain is from xyn10B from Clostridium stercorarium. In further embodiments, the one or more biomass polymer-degrading enzymes are a protein containing a cellobiohydrolase catalytic domain, a beta-glucosidase, and a protein containing a cellulase catalytic domain. In further embodiments, one or more of the following is true, the cellobiohydrolase catalytic domain is from cel6A from Cellvibrio japonicus, the beta-glucosidase is cel3B from Cellvibrio japonicus, or the cellulase catalytic domain is from cel from Bacillus sp. D04. In certain embodiments, the biomass polymer is mannan. In further embodiments, the one or more biomass polymer-degrading enzymes are an endomannanase, an exomannanase, and an alpha-galactosidase. In further embodiments, one or more of the following is true, the endomannanase is Man26A from Cellvibrio japonicus, the exomannanase is Man5D from Cellvibrio japonicus, or the alpha-galactosidase is Aga27A from Cellvibrio japonicus.

In another aspect, the invention includes a method for producing fatty alcohols from a biomass polymer, including providing a host cell, wherein the host cell contains one or more recombinant nucleic acids encoding a thioesterase, a fatty acyl-coA synthetase, a fatty alcohol-forming fatty acyl-coA reductase, and one or more biomass polymer-degrading enzymes, wherein the one or more biomass polymer-degrading enzymes are secreted from the host cell, culturing the host cell in a medium to form a culture such that the one or more recombinant nucleic acids are expressed in the cell, wherein the medium contains a biomass polymer as a carbon source for the host cell, and extracting fatty alcohols from the culture. In certain embodiments the host cell contains an endogenous nucleic acid encoding a fatty acyl-coA dehydrogenase. In certain embodiments, the host cell is modified such that expression of the fatty acyl-coA dehydrogenase is attenuated relative to the level of expression in a non-modified cell. In other embodiments, the host cell is a bacterial cell, a fungal cell, a cyanobacterial cell, a plant, animal, or human cell. In further embodiments, the host cell is an E. coli cell or a yeast cell. In certain embodiments, one or more of the following is true, the thioesterase is ltesA from E. coli, the fatty acyl-coA synthetase is fadD from E. coli, or the fatty alcohol-forming fatty acyl-coA reductase is mfar1 from Mus musculus. In certain embodiments, the biomass polymer is hemicellulose. In certain embodiments, the hemicellulose is xylan. In certain embodiments, the one or more biomass polymer-degrading enzymes are a xylanase and a protein containing an endoxylanase catalytic domain. In a further embodiment, the xylanase is xsa from Bacteroides ovatus or Gly43F from Cellvibrio japonicus. In another embodiment, the endoxylanase catalytic domain is from xyn10B from Clostridium stercorarium. In certain embodiments, the biomass polymer is cellulose. In further embodiments, the one or more biomass polymer-degrading enzymes are a protein containing a cellobiohydrolase catalytic domain, a beta-glucosidase, and a protein containing a cellulase catalytic domain. In further embodiments, one or more of the following is true, the cellobiohydrolase catalytic domain is from cel6A from Cellvibrio japonicus, the beta-glucosidase is cel3B from Cellvibrio japonicus, or the cellulase catalytic domain is from cel from Bacillus sp. D04. In certain embodiments, the biomass polymer is mannan. In further embodiments, the one or more biomass polymer-degrading enzymes are an endomannanase, an exomannanase, and an alpha-galactosidase. In further embodiments, one or more of the following is true, the endomannanase is Man26A from Cellvibrio japonicus, the exomannanase is Man5D from Cellvibrio japonicus, or the alpha-galactosidase is Aga27A from Cellvibrio japonicus.

In another aspect, the invention includes a genetically modified host cell, containing one or more recombinant nucleic acids encoding a thioesterase, a fatty acyl-coA synthetase, a fatty alcohol-forming fatty acyl-coA reductase, and one or more biomass polymer-degrading enzymes, wherein the one or more biomass polymer-degrading enzymes are secretory enzymes. In certain embodiments the host cell contains an endogenous nucleic acid encoding a fatty acyl-coA dehydrogenase. In certain embodiments, the host cell is modified such that expression of the fatty acyl-coA dehydrogenase is attenuated relative to the level of expression in a non-modified cell. In other embodiments, the host cell is a bacterial cell, a fungal cell, a cyanobacterial cell, a plant, animal, or human cell. In further embodiments, the host cell is an E. coli cell or a yeast cell. In certain embodiments, one or more of the following is true, the thioesterase is ltesA from E. coli, the fatty acyl-coA synthetase is fadD from E. coli, or the fatty alcohol-forming fatty acyl-coA reductase is mfar1 from Mus musculus. In certain embodiments, the host cell also contains recombinant nucleic acid encoding a naturally secreted protein, wherein the secreted protein is fused to the one or more biomass polymer-degrading enzymes. In further embodiments, the naturally secreted protein is OsmY from E. coli. In certain embodiments, the one or more biomass polymer-degrading enzymes are a xylanase and a protein containing an endoxylanase catalytic domain. In a further embodiment, the xylanase is xsa from Bacteroides ovatus or Gly43F from Cellvibrio japonicus. In another embodiment, the endoxylanase catalytic domain is from xyn10B from Clostridium stercorarium. In further embodiments, the one or more biomass polymer-degrading enzymes are a protein containing a cellobiohydrolase catalytic domain, a beta-glucosidase, and a protein containing an cellulase catalytic domain. In further embodiments, one or more of the following is true, the cellobiohydrolase catalytic domain is from cel6A from Cellvibrio japonicus, the beta-glucosidase is cel3B from Cellvibrio japonicus, or the cellulase catalytic domain is from cel from Bacillus sp. D04. In certain embodiments, the biomass polymer is mannan. In further embodiments, the one or more biomass polymer-degrading enzymes are an endomannanase, an exomannanase, and an alpha-galactosidase. In further embodiments, one or more of the following is true, the endomannanase is Man26A from Cellvibrio japonicus, the exomannanase is Man5D from Cellvibrio japonicus, or the alpha-galactosidase is Aga27A from Cellvibrio japonicus.

In another aspect, the invention includes a method for producing fatty aldehydes from a biomass polymer, including providing a host cell, wherein the host cell contains one or more recombinant nucleic acids encoding a thioesterase, a fatty acyl-coA synthetase, a fatty acyl-coA reductase, and one or more biomass polymer-degrading enzymes, wherein the one or more biomass polymer-degrading enzymes are secreted from the host cell, culturing the host cell in a medium to form a culture such that the one or more recombinant nucleic acids are expressed in the cell, wherein the medium contains a biomass polymer as a carbon source for the host cell, and extracting fatty aldehydes from the culture. In certain embodiments the host cell contains an endogenous nucleic acid encoding a fatty acyl-coA dehydrogenase. In certain embodiments, the host cell is modified such that expression of the fatty acyl-coA dehydrogenase is attenuated relative to the level of expression in a non-modified cell. In other embodiments, the host cell is a bacterial cell, a fungal cell, a cyanobacterial cell, a plant, animal, or human cell. In further embodiments, the host cell is an E. coli cell or a yeast cell. In certain embodiments, one or more of the following is true, the thioesterase is ltesA from E. coli, the fatty acyl-coA synthetase is fadD from E. coli, or the fatty acyl-coA reductase is acr1 from Acinetobacter baylyi. In certain embodiments, the biomass polymer is hemicellulose. In certain embodiments, the hemicellulose is xylan. In certain embodiments, the one or more biomass polymer-degrading enzymes are a xylanase and a protein containing an endoxylanase catalytic domain. In a further embodiment, the xylanase is xsa from Bacteroides ovatus or Gly43F from Cellvibrio japonicus. In another embodiment, the endoxylanase catalytic domain is from xyn10B from Clostridium stercorarium. In certain embodiments, the biomass polymer is cellulose. In further embodiments, the one or more biomass polymer-degrading enzymes are a protein containing a cellobiohydrolase catalytic domain, a beta-glucosidase, and a protein containing a cellulase catalytic domain. In further embodiments, one or more of the following is true, the cellobiohydrolase catalytic domain is from cel6A from Cellvibrio japonicus, the beta-glucosidase is cel3B from Cellvibrio japonicus, or the cellulase catalytic domain is from cel from Bacillus sp. D04. In certain embodiments, the biomass polymer is mannan. In further embodiments, the one or more biomass polymer-degrading enzymes are an endomannanase, an exomannanase, and an alpha-galactosidase. In further embodiments, one or more of the following is true, the endomannanase is Man26A from Cellvibrio japonicus, the exomannanase is Man5D from Cellvibrio japonicus, or the alpha-galactosidase is Aga27A from Cellvibrio japonicus.

In another aspect, the invention includes a genetically modified host cell, containing one or more recombinant nucleic acids encoding a thioesterase, a fatty acyl-coA synthetase, a fatty acyl-coA reductase, and one or more biomass polymer-degrading enzymes, wherein the one or more biomass polymer-degrading enzymes are secretory enzymes. In certain embodiments the host cell contains an endogenous nucleic acid encoding a fatty acyl-coA dehydrogenase. In certain embodiments, the host cell is modified such that expression of the fatty acyl-coA dehydrogenase is attenuated relative to the level of expression in a non-modified cell. In other embodiments, the host cell is a bacterial cell, a fungal cell, a cyanobacterial cell, a plant, animal, or human cell. In further embodiments, the host cell is an E. coli cell or a yeast cell. In certain embodiments, one or more of the following is true, the thioesterase is ltesA from E. coli, the fatty acyl-coA synthetase is fadD from E. coli, or the fatty acyl-coA reductase is acr1 from Acinetobacter baylyi. In certain embodiments, the host cell also contains recombinant nucleic acid encoding a naturally secreted protein, wherein the secreted protein is fused to the one or more biomass polymer-degrading enzymes. In further embodiments, the naturally secreted protein is OsmY from E. coli. In certain embodiments, the one or more biomass polymer-degrading enzymes are a xylanase and a protein containing an endoxylanase catalytic domain. In a further embodiment, the xylanase is xsa from Bacteroides ovatus or Gly43F from Cellvibrio japonicus. In another embodiment, the endoxylanase catalytic domain is from xyn10B from Clostridium stercorarium. In further embodiments, the one or more biomass polymer-degrading enzymes are a protein containing a cellobiohydrolase catalytic domain, a beta-glucosidase, and a protein containing an cellulase catalytic domain. In further embodiments, one or more of the following is true, the cellobiohydrolase catalytic domain is from cel6A from Cellvibrio japonicus, the beta-glucosidase is cel3B from Cellvibrio japonicus, or the cellulase catalytic domain is from cel from Bacillus sp. D04. In certain embodiments, the biomass polymer is mannan. In further embodiments, the one or more biomass polymer-degrading enzymes are an endomannanase, an exomannanase, and an alpha-galactosidase. In further embodiments, one or more of the following is true, the endomannanase is Man26A from Cellvibrio japonicus, the exomannanase is Man5D from Cellvibrio japonicus, or the alpha-galactosidase is Aga27A from Cellvibrio japonicus.

In another aspect, the invention includes methods and host cells for the utilization of mannan, wherein the biomass polymer is mannan and the biomass polymer-degrading enzymes are endo-mannanase, exomannanase, and alpha-galactosidase. A further embodiment includes methods and host cells for the production of fatty acid ethyl esters, fatty alcohols, or fatty aldehydes from mannan.

In another aspect, the invention includes methods and host cells for producing fatty acid esters. In one embodiment, the invention includes methods and host cells for the production of fatty ethyl esters from a biomass polymer. In one embodiment, the host cell contains one or more recombinant nucleic acids encoding a thioesterase, a fatty acyl-coA synthetase, fatty alcohol-forming fatty acyl-coA reductase, and an acyltransferase.

In another aspect, the invention includes methods of producing fatty acid ethyl esters, fatty alcohols, or fatty aldehydes from sunlight. In one embodiment, an organism capable of using sunlight as a carbon source is genetically engineered to contain the enzymatic pathways to produce fatty acid ethyl esters, fatty alcohols, or fatty aldehydes as described above in other aspects of the invention. In another embodiment, the host cells of the above aspects of the invention are further genetically engineered to contain enzymatic pathways that allow the host cell to utilize sunlight as a carbon source and to produce fatty acid ethyl esters, fatty alcohols, or fatty aldehydes directly from sunlight.

### BRIEF DESCRIPTION OF THE DRAWINGS

FIG. 1 shows engineered pathways for production of fatty acid-derived molecules from hemicelluloses or glucose and depiction of the synthetic operons used in this study (FIG. 1A). Flux through native E. coli metabolic pathways (lines with black arrows) was increased to improve production of free fatty acids and acyl-CoAs by removal of the acetate-forming reactions (knockouts are pta, ackA, and poxB). Flux was further increased by eliminating fatty acid and fatty acyl-CoA catabolism by β-oxidation. Various non-native E. coli products were produced from non-native pathways (lines with gray arrows) including fatty acid ethyl esters, alcohols, and aldehydes. The alcohols and aldehydes can be produced directly from fatty acyl-CoAs (using mFar1 or acr1, respectively), while the esters required introduction of an ethanol production pathway (encoded by pdc and adhB). Finally, demonstration of consolidated bioprocessing was achieved by expressing and secreting an endoxylanase, Xyn10B, and a xylanase from C. stercorarium allowing our biofuel-producing E. coli to utilize hemicellulose as a carbon source. Over-expressed genes or operons are indicated; triangles represent the lacUV5 promoter. FIG. 1B shows an example of a fatty acid ethyl ester pathway, FIG. 1C shows an example of a fatty alcohol pathway, FIG. 1D shows an example of a fatty aldehyde pathway, and FIG. 1E shows an example of a fatty acid ester/wax ester pathway. Abbreviations: Pyr—pyruvate; AcAld—acetaldehyde; EtOH—ethanol.

FIG. 2 shows total free fatty acid production by engineered E. coli strains. Overexpressed and knocked out genes are indicated. WT: wild-type DH1; LT: thioesterase; LT-ΔfadD: ΔfadD, LtesA; LT-ΔfadE: ΔfadE, LtesA; LT-ΔfadD-ΔACE: Δpta, ΔpoxB, ΔackA, ΔfadD, LtesA.

FIG. 3 shows biodiesel equivalent production by various strains. HE-LAAP: ΔfadE, LtesA, atfA, pdc, adhB; faa2: ΔfadE, LtesA, atfA, pdc, adhB, faa2; HE-atf″: ΔfadE, LtesA, atfA, pdc, adhB, fadDm2; A1A: ΔfadE, LtesA, atfA, pdc, adhB, fadDm1; A2A: ΔfadE, LtesA, 2 copies of atfA, pdc, adhB, fadDm1.

FIG. 4 shows fatty alcohol production by strains KS5 and KS11. Detection of the C12 to C18 fatty alcohols was achieved. KS5: ΔfadE, mFar1; KS11: ΔfadE, acr1.

FIG. 5A-FIG. 5C show growth of xylan utilization strains (FIGS. 5A and 5B) and FAEE production (FIG. 5C). In FIG. 5A, genes encoding endo-xylanase or beta-xylosidase were transformed into E. coli individually or on one plasmid to test for utilization of xylan. In FIG. 5B, blue diamonds, GB-X, is BL21 background expressing xylanase xynB from plasmid pGB-X; Green triangles, GB-XX, is BL21 background expressing xylanase xynB and endoxylanase xsa from plasmid pGB-XX. Both strains are grown in 0.2% xylan M9 minimal media. FIG. 5C shows FAEE production: HE-XH: DH1, ΔfadE, expressing xynB, xsa, LtesA, atfA, pdc, and adhB, grown in 0.2% xylose; PE2-XX: DH1, ΔfadE, Δpta, ΔpoxB, ΔackA, expressing xynB, xsa, LtesA, atfA, pdc, and adhB, grown in 0.2% xylose and 2% xylan.

FIG. 6 shows free fatty acid chain length distribution in LtesA-expressing strains.

FIG. 7 shows growth of recombinant E. coli on carboxymethyl cellulose.

FIG. 8 shows growth of co-cultures of recombinant E. coli on galactomannan.

FIG. 9A-FIG. 9C show production of fatty acid esters from E. coli. FIG. 9A shows production of tetradecanoate hexadecylester. FIG. 9B shows production of hexdecanoate hexadecylester. FIG. 9C shows production of hexdecanoate octadecylester.

FIG. 10 shows a comparison of growth of E. coli containing a plasmid with OsmY-XynB and OsmY-Xsa and E. coli containing a plasmid with OsmY-XynB and untagged Xsa in media containing xylan as the sole carbon source.

FIG. 11 shows growth of E. coli expressing a cellulase and a beta-glucosidase on regenerated amorphous cellulose (RAC). Control E. coli did not express a cellulase.

FIG. 12 shows growth of E. coli expressing OsmY-XynB and Gly43F in media containing xylan or xylose as the sole carbon source.

### DETAILED DESCRIPTION OF THE INVENTION

The present disclosure relates to consolidated bioprocessing methods and host cells. In certain embodiments, the host cells are capable of producing biodiesel equivalents and other fatty acid derivatives. In other embodiments, the host cells have the ability to directly convert biomass polymers or sunlight into biodiesel equivalents and other fatty acid derivatives. In one aspect, the invention provides a method for producing biodiesel equivalents and other fatty acid derivatives from a biomass polymer including providing a genetically engineered host cell, culturing the host cell in a medium containing a carbon source such that recombinant nucleic acids in the cell are expressed, and extracting biodiesel equivalents and other fatty acid derivatives from the culture.

Host Cells of the Invention

“Host cell” and “host microorganism” are used interchangeably herein to refer to a living biological cell that can be transformed via insertion of recombinant DNA or RNA. Such recombinant DNA or RNA can be in an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

Any prokaryotic or eukaryotic host cell may be used in the present invention so long as it remains viable after being transformed with a sequence of nucleic acids. In preferred embodiments, the host microorganism is bacterial, and in some embodiments, the bacteria are E. coli. In other embodiments, the bacteria are cyanobacteria. Additional examples of bacterial host cells include, without limitation, those species assigned to the Escherichia, Enterobacter, Azotobacter, Erwinia, Bacillus, Pseudomonas, Klebsiella, Proteus, Salmonella, Serratia, Shigella, Rhizobia, Vitreoscilla, Synechococcus, Synechocystis, and Paracoccus taxonomical classes. Preferably, the host cell is not adversely affected by the transduction of the necessary nucleic acid sequences, the subsequent expression of the proteins (i.e., enzymes), or the resulting intermediates.

Suitable eukaryotic cells include, but are not limited to, fungal, plant, insect or mammalian cells. Suitable fungal cells are yeast cells, such as yeast cells of the Saccharomyces genus. In some embodiments the eukaryotic cell is from algae, e.g., Chlamydomonas reinhardtii, Scenedesmus obliquus, Chlorella vulgaris, or Dunaliella salina.

The host cells of the present invention are genetically modified in that recombinant nucleic acids have been introduced into the host cells, and as such the genetically modified host cells do not occur in nature. The suitable host cell is one capable of expressing one or more nucleic acid constructs encoding one or more enzymes capable of catalyzing a desired biosynthetic reaction, In preferred embodiments, the one or more enzymes include, but are not limited to, a thioesterase, a fatty acyl coA synthetase, an acyl transferase, an alcohol dehydrogenase, a pyruvate decarboxylase, one or more biomass polymer-degrading enzymes, a fatty alcohol-forming fatty acyl-coA reductase, or a fatty acyl-coA reductase. In preferred embodiments, the one or more enzymes are capable of catalyzing reactions which lead to the production of biodiesel equivalents or other fatty acid derivatives.

“Recombinant nucleic acid” or “heterologous nucleic acid” as used herein refers to a polymer of nucleic acids wherein at least one of the following is true: (a) the sequence of nucleic acids is foreign to (i.e., not naturally found in) a given host microorganism; (b) the sequence may be naturally found in a given host microorganism, but is present in an unnatural (e.g., greater than expected) amount; or (c) the sequence of nucleic acids comprises two or more subsequences that are not found in the same relationship to each other in nature. For example, regarding instance (c), a recombinant nucleic acid sequence will have two or more sequences from unrelated genes arranged to make a new functional nucleic acid. Specifically, the present invention describes the introduction of an expression vector into a host cell, wherein the expression vector contains a nucleic acid sequence coding for an enzyme that is not normally found in a host cell or contains a nucleic acid coding for an enzyme that is normally found in a cell but is under the control of different regulatory sequences. With reference to the host cell's genome, then, the nucleic acid sequence that codes for the enzyme is recombinant.

In some embodiments, the host cell naturally produces any of the precursors for the production of the fatty acid-derived compounds. These genes encoding the desired enzymes may be heterologous to the host cell, or these genes may be endogenous to the host cell but are operatively linked to heterologous promoters and/or control regions which result in higher expression of the gene(s) in the host cell. In other embodiments, the host cell does not naturally produce the desired fatty acid molecule and comprises heterologous nucleic acid constructs capable of expressing one or more genes necessary for producing those molecules.

“Endogenous” as used herein with reference to a nucleic acid molecule or polypeptide and a particular cell or microorganism refers to a nucleic acid sequence or peptide that is in the cell and was not introduced into the cell using recombinant engineering techniques, for example, a gene that was present in the cell when the cell was originally isolated from nature.

Each of the desired enzymes capable of catalyzing the desired reaction can be native or heterologous to the host cell. Where the enzyme is native to the host cell, the host cell is optionally genetically modified to modulate expression of the enzyme. This modification can involve the modification of the chromosomal gene encoding the enzyme in the host cell or introduction of a nucleic acid construct encoding the gene of the enzyme into the host cell. One of the effects of the modification is the expression of the enzyme is modulated in the host cell, such as the increased expression of the enzyme in the host cell as compared to the expression of the enzyme in an unmodified host cell. Alternatively, modification of expression of an enzyme may result in decreased expression of the enzyme in the host cell as compared to expression of the enzyme in an unmodified cell. For example, a host cell may contain a native nucleic acid that encodes a fatty acyl-coA dehydrogenase. In some aspects of the invention, the host cell may be genetically modified such that expression of the fatty acyl-coA dehydrogenase is reduced or attenuated relative to its level of expression in an unmodified host cell.

Genetic modifications include any type of modification and specifically include modifications made by recombinant technology and/or by classical mutagenesis. As used herein, genetic modifications which result in a decrease in gene expression, in the function of the gene, or in the function of the gene product (i.e., the protein encoded by the gene) can be referred to as inactivation (complete or partial), deletion, interruption, blockage, silencing, or down-regulation, or attenuation of expression of a gene. For example, a genetic modification in a gene which results in a decrease in the function of the protein encoded by such gene, can be the result of a complete deletion of the gene (i.e., the gene does not exist, and therefore the protein does not exist), a mutation in the gene which results in incomplete or no translation of the protein (e.g., the protein is not expressed), or a mutation in the gene which decreases or abolishes the natural function of the protein (e.g., a protein is expressed which has decreased or no enzymatic activity or action). More specifically, reference to decreasing the action or activity of enzymes discussed herein generally refers to any genetic modification in the microorganism in question which results in decreased expression and/or functionality (biological activity) of the enzymes and includes decreased activity of the enzymes (e.g., specific activity), increased inhibition or degradation of the enzymes, as well as a reduction or elimination of expression of the enzymes. For example, the action or activity of an enzyme of the present invention can be decreased by blocking or reducing the production of the enzyme, reducing enzyme activity, or inhibiting the activity of the enzyme. Combinations of some of these modifications are also possible. Blocking or reducing the production of an enzyme can include placing the gene encoding the enzyme under the control of a promoter that requires the presence of an inducing compound in the growth medium. By establishing conditions such that the inducer becomes depleted from the medium, the expression of the gene encoding the enzyme (and therefore, of enzyme synthesis) could be turned off. Blocking or reducing the activity of an enzyme could also include using an excision technology approach similar to that described in U.S. Pat. No. 4,743,546. To use this approach, the gene encoding the enzyme of interest is cloned between specific genetic sequences that allow specific, controlled excision of the gene from the genome. Excision could be prompted by, for example, a shift in the cultivation temperature of the culture, as in U.S. Pat. No. 4,743,546, or by some other physical or nutritional signal.

“Genetically engineered” or “genetically modified” refer to any recombinant DNA or RNA method used to create a prokaryotic or eukaryotic host cell that expresses a protein at elevated levels, at lowered levels, or in a mutated form. In other words, the host cell has been transfected, transformed, or transduced with a recombinant polynucleotide molecule, and thereby been altered so as to cause the cell to alter expression of a desired protein. Methods and vectors for genetically engineering host cells are well known in the art; for example, various techniques are illustrated in Current Protocols in Molecular Biology, Ausubel et al., eds. (Wiley & Sons, New York, 1988, and quarterly updates). Genetically engineering techniques include but are not limited to expression vectors, targeted homologous recombination and gene activation (see, for example, U.S. Pat. No. 5,272,071), and trans-activation by engineered transcription factors (see, for example, Segal et al., 1999, Proc Natl Acad Sci USA 96(6):2758-63).

Genetic modifications that result in an increase in gene expression or function can be referred to as amplification, overproduction, overexpression, activation, enhancement, addition, or up-regulation of a gene. More specifically, reference to increasing the action (or activity) of enzymes or other proteins discussed herein generally refers to any genetic modification in the microorganism in question which results in increased expression and/or functionality (biological activity) of the enzymes or proteins and includes higher activity of the enzymes (e.g., specific activity or in vivo enzymatic activity), reduced inhibition or degradation of the enzymes, and overexpression of the enzymes. For example, gene copy number can be increased, expression levels can be increased by use of a promoter that gives higher levels of expression than that of the native promoter, or a gene can be altered by genetic engineering or classical mutagenesis to increase the biological activity of an enzyme. Combinations of some of these modifications are also possible.

In general, according to the present invention, an increase or a decrease in a given characteristic of a mutant or modified enzyme (e.g., enzyme activity) is made with reference to the same characteristic of a wild-type (i.e., normal, not modified) enzyme that is derived from the same organism (from the same source or parent sequence), which is measured or established under the same or equivalent conditions. Similarly, an increase or decrease in a characteristic of a genetically modified microorganism (e.g., expression and/or biological activity of a protein, or production of a product) is made with reference to the same characteristic of a wild-type microorganism of the same species, and preferably the same strain, under the same or equivalent conditions. Such conditions include the assay or culture conditions (e.g., medium components, temperature, pH, etc.) under which the activity of the protein (e.g., expression or biological activity) or other characteristic of the microorganism is measured, as well as the type of assay used, the host microorganism that is evaluated, etc. As discussed above, equivalent conditions are conditions (e.g., culture conditions) which are similar, but not necessarily identical (e.g., some conservative changes in conditions can be tolerated), and which do not substantially change the effect on microbe growth or enzyme expression or biological activity as compared to a comparison made under the same conditions.

Preferably, a genetically modified host cell that has a genetic modification that increases or decreases the activity of a given protein (e.g., an enzyme) has an increase or decrease, respectively, in the activity (e.g., expression, production and/or biological activity) of the protein, as compared to the activity of the wild-type protein in a wild-type microorganism, of at least about 5%, and more preferably at least about 10%, and more preferably at least about 15%, and more preferably at least about 20%, and more preferably at least about 25%, and more preferably at least about 30%, and more preferably at least about 35%, and more preferably at least about 40%, and more preferably at least about 45%, and more preferably at least about 50%, and more preferably at least about 55%, and more preferably at least about 60%, and more preferably at least about 65%, and more preferably at least about 70%, and more preferably at least about 75%, and more preferably at least about 80%, and more preferably at least about 85%, and more preferably at least about 90%, and more preferably at least about 95%, or any percentage, in whole integers between 5% and 100% (e.g., 6%, 7%, 8%, etc.). The same differences are preferred when comparing the activity of an isolated modified nucleic acid molecule or protein directly to the activity of an isolated wild-type nucleic acid molecule or protein (e.g., if the comparison is done in vitro as compared to in vivo).

In another aspect of the invention, a genetically modified host cell that has a genetic modification that increases or decreases the activity of a given protein (e.g., an enzyme) has an increase or decrease, respectively, in the activity (e.g., expression, production and/or biological activity) of the protein, as compared to the activity of the wild-type protein in a wild-type microorganism, of at least about 2-fold, and more preferably at least about 5-fold, and more preferably at least about 10-fold, and more preferably about 20-fold, and more preferably at least about 30-fold, and more preferably at least about 40-fold, and more preferably at least about 50-fold, and more preferably at least about 75-fold, and more preferably at least about 100-fold, and more preferably at least about 125-fold, and more preferably at least about 150-fold, or any whole integer increment starting from at least about 2-fold (e.g., 3-fold, 4-fold, 5-fold, 6-fold, etc.).

Enzymes and Constructs Encoding Thereof of the Invention

Enzymes of the invention include any enzymes involved in pathways that lead directly or indirectly to the production of biodiesel equivalents or other fatty acid derivatives in a host cell. Enzymes of the invention may, for example, catalyze the production of intermediates or substrates for further reactions leading to the production of biodiesel equivalents or other fatty acid derivatives in a host cell. In some embodiments, enzymes of the invention are secretory enzymes. Enzymes of the invention include, without limitation, a thioesterase, a fatty acyl coA synthetase, an acyl transferase, an alcohol dehydrogenase, a pyruvate decarboxylase, a fatty alcohol-forming fatty acyl-coA reductase, a fatty acyl-coA reductase, or an acyl-coA dehydrogenase, and one or more biomass polymer-degrading enzymes, such as a xylanase, an endoxylanase, a cellobiohydrolase, a beta-glucosidase, a cellulase, an endo-mannanase, an exomannanase, or an alpha-galactosidase.

A thioesterase includes any enzyme that exhibits esterase activity (splitting of an ester into acid and alcohol, in the presence of water) specifically at a thiol group. For example, a thioesterase may be ltesA from E. coli (GenBank Accession AAC73596). Other thioesterases include, without limitation, those listed in Table 1 below.

TABLE 1

| GenBank | | | |
|---|---|---|---|
| Accession No. | Organism Source | Gene | Specificity |
| AAC73596 | E. coli | tesAw/o leader | C18:1 |
| | | sequence | |
| Q41635 | U. california | fatB | C12:0 |
| Q39513; | C. hookeriania | fatB2 | C8:0-10:0 |
| AAC49269 | C. hookeriania | fatB3 | C14:0-16:0 |
| Q39473 | C. camphorum | fatB | C14:0 |
| CAA85388 | A. thaliana | fatB(M1T) | C16:1 |
| NP 189147; NP | A. thaliana | fatA | C18:1 |
| 193041 | | | |
| CAC39106 | B. japonicum | fatA | C18:1 |
| AAC72883 | C. hookeriania | fatA | C18:1 |

A fatty acyl coA synthetase includes any enzyme that catalyzes the chemical reaction of acetyl-CoA+n malonyl-CoA+2n NADH+2n NADPH+4n H⁺ [symbol] long-chain-acyl-CoA+n CoA+n CO2+2n NAD⁺+2n NADP⁺. This enzyme is also known as a fatty acid coA ligase. For example, a fatty acyl-coA synthetase may be fadD from E. coli (GenBank Accession No. AP_{—}002424). In other embodiments, a fatty acyl-coA synthetase may be faa1 (Accession No. NP_{—}014962.1), faa2 (Accession No. NP_{—}010931.1), faa3 (Accession No. NP_{—}012257.1), or faa4 (Accession No. NP_{—}013974.1), all of which are from S. cerevisiae).

An acyl-transferase includes any type of transferase enzyme which acts upon acyl groups. For example, an acyl-transferase may be the wax ester synthase atfA (wax-dgat) from Acinetobacter sp. ADP1 (GenBank Accession No. AF529086). In another embodiment, the acyl-transferase may be dgat from S. cerevisiae.

An alcohol dehydrogenase includes any enzyme that facilitates the interconversion between alcohols and aldehydes or ketones with the reduction of NAD+ to NADH. For example, an alcohol dehydrogenase may be adhB from Zymomonas mobilis.

A pyruvate decarboxylase includes any homotetrameric enzyme that catalyses the decarboxylation of pyruvic acid to acetaldehyde and carbon dioxide. For example, a pyruvate decarboxylase may be pdc from Zymomonas mobilis.

A fatty alcohol-forming fatty acyl-coA reductase is any enzyme that reduces a fatty acyl-coA to form fatty alcohols. For example, a fatty alcohol forming fatty acyl-coA may be mFar1 from Mus musculus. In other embodiments, it may be BmFAR from Bombyx mori (GenBank Accession No. BAC79425), mFAR2 from Mus musculus, or hFAR from human.

A fatty acyl-coA reductase is any enzyme that catalyzes the chemical reaction of a long-chain aldehyde+CoA+NADP⁺ [symbol] a long-chain acyl-CoA+NADPH+H⁺. For example, a fatty acyl-coA reductase may be acr1 from Acinetobacter sp. ADP1 (GenBank Accession No. YP_{—}047869). In other embodiments, the fatty acyl-coA reductase may be yqhD from E. coli (GenBank Accession No. AP_{—}003562).

An acyl-coA dehydrogenase is any enzyme whose action results in the introduction of a trans double-bond between C2 and C3 of a acyl-CoA thioester substrate. For example, an acyl-coA dehydrogenase may be fadE from E. coli.

Biomass polymer-degrading enzymes include any enzymes able to degrade any biomass polymer. “A biomass polymer” as described herein is any polymer contained in biological material. The biological material may be living or dead. A biomass polymer includes, for example, cellulose, xylan, hemicellulose, lignin, mannan, and other materials commonly found in biomass. Non-limiting examples of sources of a biomass polymer include grasses (e.g., switchgrass, Miscanthus), rice hulls, bagasse, cotton, jute, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe).

Biomass polymer-degrading enzymes may include, without limitation, a xylanase, such as xsa from Bacteroides ovatus or Gly43F from Cellvibrio japonicus, an endoxylanase catalytic domain, such as from xyn10B from Clostridium stercorarium, a cellobiohydrolase catalytic domain, such as from cel6A from Cellvibrio japonicus, a beta-glucosidase, such as cel3B from Cellvibrio japonicus, a cellulase catalytic domain, such as from cel from Bacillus sp. D04, an endomannanase catalytic domain, such as Man26A from Cellvibrio japonicus, an exomannase, such as Man5D from Cellvibrio japonicus, or an alpha-galactosidase, such as Aga27A from Cellvibrio japonicus.

Additional examples of enzymes of the invention may be found, without limitation, in Kalscheuer, Stölting, and Steinbüchel Microbiology(2006) 12 2529-2539; Ingram et al Appl Environ Microbiol (1987) 53 2420-2425; WO 2008/100251; WO 2008/119082; WO 2007/136762; and WO 2009/009391.

The enzymes described herein can be readily replaced using a homologous enzyme thereof. “Homologous enzymes” as used herein refer to enzymes that have a polypeptide sequence that is at least 70%, at least 75%, at least 80%, at least 85%, at least 90%, at least 95% or at least 99% identical to any one of the enzymes described in this specification or in a cited reference. Homologous enzymes retain amino acid residues that are recognized as conserved for the enzyme. Homologous enzymes may have non-conserved amino acid residues replaced or found to be of a different amino acid, or amino acid(s) inserted or deleted, as long as they do not affect or have insignificant effect on the enzymatic activity of the homologous enzyme. Homologous enzyme have an enzymatic activity that is essentially the same as the enzymatic activity of any one of the enzymes described in this specification or in a cited reference in that it will catalyze the same reaction. The specific activity of the enzyme may be increased or decreased. Homologous enzymes may be found in nature or be an engineered mutant thereof. The enzymes described herein can also be replaced by an isozyme, an enzyme that may differ in amino acid sequence but that catalyzes the same chemical reaction.

The nucleic acid constructs of the present invention include nucleic acid sequences encoding one or more of the subject enzymes. The nucleic acid of the subject enzymes are operably linked to promoters and optional control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; internucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. “Expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host microorganism. Optionally, the expression vector also comprises materials to aid in achieving entry of the nucleic acid into the host microorganism, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host microorganism and replicated therein. Preferred expression vectors are plasmids, particularly those with restriction sites that have been well-documented and that contain the operational elements preferred or required for transcription of the nucleic acid sequence. Such plasmids, as well as other expression vectors, are well known to those of ordinary skill in the art.

Incorporation of the individual nucleic acid sequences may be accomplished through known methods that include, for example, the use of restriction enzymes (such as BamHI, EcoRI, Hhal, Xhol, Xmal, and so forth) to cleave specific sites in the expression vector, e.g., plasmid. The restriction enzyme produces single-stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers maybe used to facilitate linking of nucleic acids sequences into an expression vector.

A series of individual nucleic acid sequences can also be combined by utilizing methods that are known to those having ordinary skill in the art (e.g., U.S. Pat. No. 4,683,195). For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other. Extension of this overlap by DNA polymerase produces a molecule in which the original sequences are “spliced” together. In this way, a series of individual nucleic acid sequences may be “spliced” together and subsequently transduced into a host cell simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli (see Shine et al. (1975) Nature 254:34 and Steitz, Biological Regulation and Development: Gene Expression (ed. R. F. Goldberger), vol. 1, p. 349, 1979, Plenum Publishing, NY).

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter (see deBoer et al. (1983) Proc Natl Acad Sci USA, 80:21-25). As will be appreciated by those of ordinary skill in the art, these and other expression vectors may be used in the present invention, and the invention is not limited in this respect.

Although any suitable expression vector may be used to incorporate the desired sequences, readily-available expression vectors include, without limitation: plasmids, such as pSC1O1, pBR322, pBBR1MCS-3, pUR, pEX, pMR1OO, pCR4, pBAD24, pUC19, and bacteriophages, such as M1 3 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

Methods of Producing and Culturing Host Cells of the Invention

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of a current to increase the permeability of cells to nucleic acid sequences) may be used to transfect the host microorganism. Also, microinjection of the nucleic acid sequences provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

The host cell is transformed with at least one expression vector. When only a single expression vector is used (without the addition of an intermediate), the vector will contain all of the necessary nucleic acid sequences.

Once the host cell has been transformed with the expression vector, the host cell is allowed to grow. Methods of the invention include culturing the host cell such that recombinant nucleic acids in the cell are expressed. For microbial hosts, this process entails culturing the cells in a suitable medium. Typically cells are grown at 35° C. in appropriate media. Preferred growth media in the present invention are common commercially prepared media such as Luria Bertani (LB) broth, Sabouraud Dextrose (SD) broth or yeast medium (YM) broth. Other defined or synthetic growth media may also be used, and the appropriate medium for growth of the particular host cell will be known by someone skilled in the art of microbiology or fermentation science.

According to some aspects of the invention, the culture media contains a carbon source for the host cell. Such a “carbon source” generally refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, etc. These include, for example, various monosaccharides, such as glucose, xylose, and arabinose, disaccharides, such as sucrose, oligosaccharides, polysaccharides, biomass polymers, such as cellulose and hemicellulose, saturated or unsaturated fatty acids, succinate, lactate, acetate, ethanol, etc., or mixtures thereof. The carbon source can additionally be a product of photosynthesis, including, but not limited to glucose.

In addition to an appropriate carbon source, fermentation media must contain suitable minerals, salts, cofactors, buffers and other components, known to those skilled in the art, suitable for the growth of the cultures and promotion of the enzymatic pathways necessary for production of fatty acid-derived molecules. Reactions may be performed under aerobic or anaerobic conditions where aerobic, anoxic, or anaerobic conditions are preferred based on the requirements of the microorganism. As the host cell grows and/or multiplies, the enzymes necessary for producing FAEEs, fatty alcohols, fatty aldehydes, and other fatty acid derivatives are expressed.

Biodiesel Equivalents and Other Fatty Acid Derivatives of the Invention

The present invention provides for the production of biodiesel equivalents and other fatty acid derivatives. The biodiesel equivalents and other fatty acid derivatives include, without limitation, fatty acid ethyl esters, fatty acid esters, wax esters, fatty alcohols, and fatty aldehydes.

The present invention provides for an isolated fatty acid-derived compound produced from the method of the present invention. Isolating the fatty acid-derived compound involves the separating at least part or all of the host cells, and parts thereof, from which the fatty acid-derived compound was produced, from the isolated fatty acid-derived compound. The isolated fatty acid derived compound may be free or essentially free of impurities formed from at least part or all of the host cells, and parts thereof. The isolated fatty acid derived compound is essentially free of these impurities when the amount and properties of the impurities remaining do not interfere in the use of the fatty acid derived compound as a fuel, such as a fuel in a combustion reaction.

The present invention also provides for a combustible composition comprising an isolated fatty acid-derived compound and cellular components, wherein the cellular components do not substantially interfere in the combustion of the composition. The cellular components include whole cells or parts thereof. The cellular components are derived from host cells which produced the fatty acid derived compound.

The fatty acid derived compounds of the present invention are useful as fuels as chemical source of energy that can be used as an alternative to petroleum-derived fuels, ethanol and the like. The fatty acid-derived compounds of the present invention are also useful in the synthesis of alkanes, alcohols, and esters for various uses as a renewable fuel. In addition, the fatty acid-derived compounds can also be used as precursors in the synthesis of therapeutics, or high-value oils, such as a cocoa butter equivalent.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

The invention having been described, the following examples are offered to illustrate the subject invention by way of illustration, not by way of limitation.

### EXAMPLES

### Example 1

### Deregulation of Fatty Acid Biosynthesis by Cytosolic Thioesterase Expression

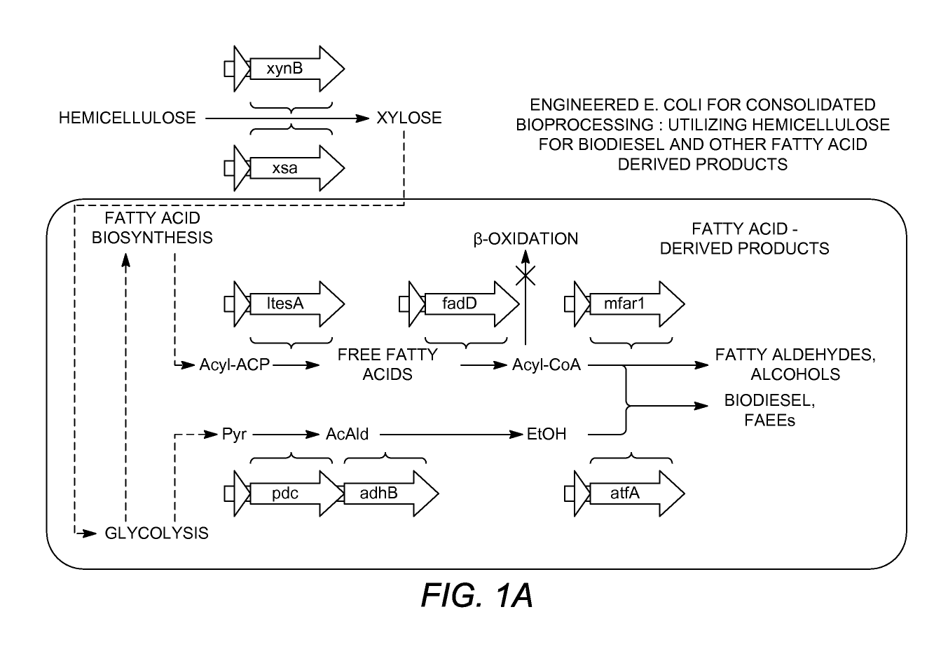

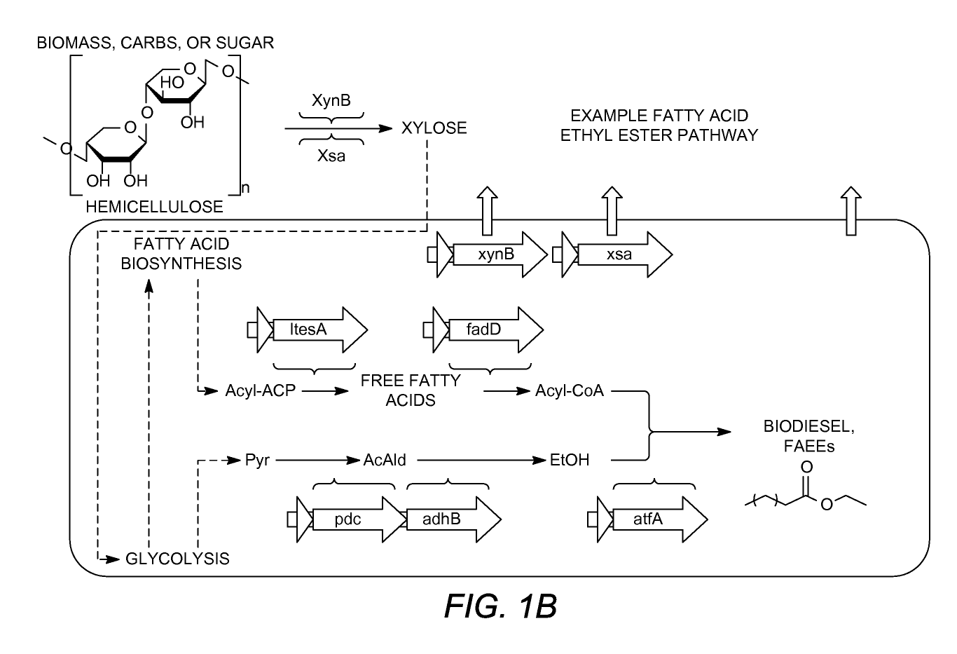

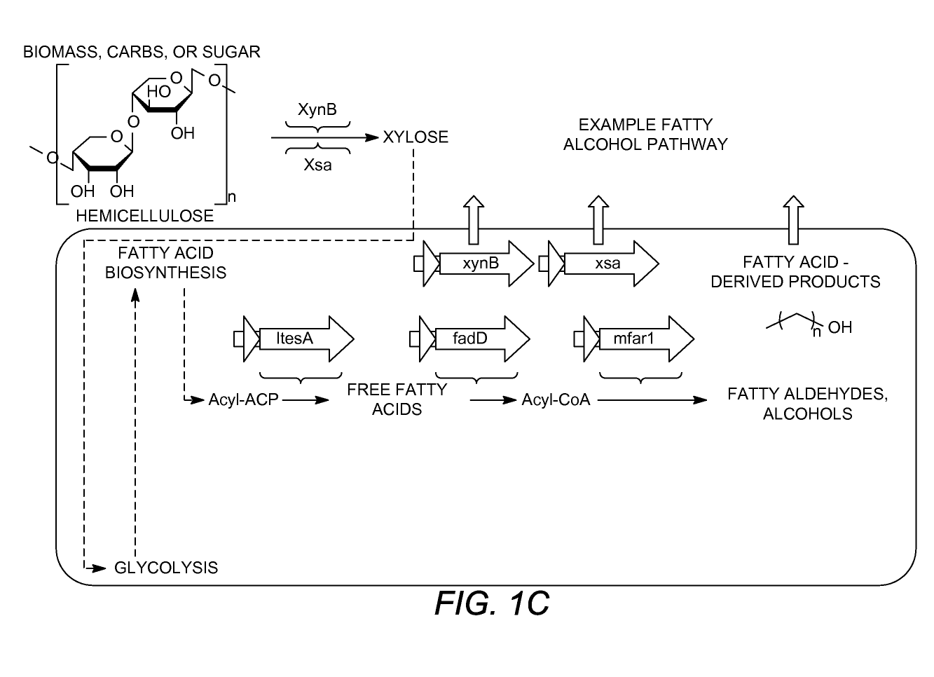

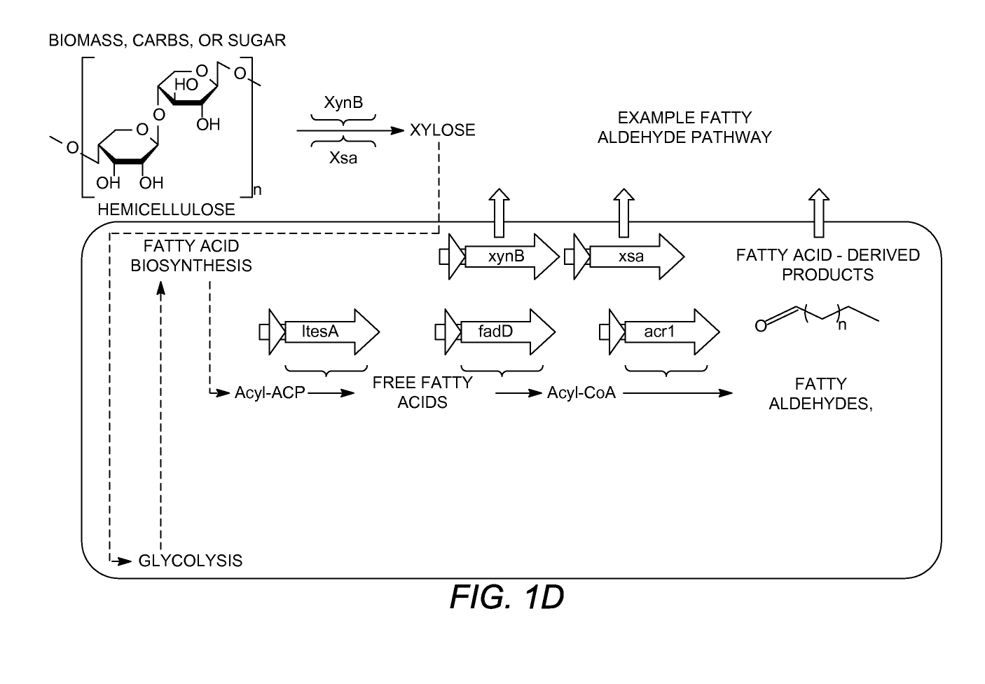

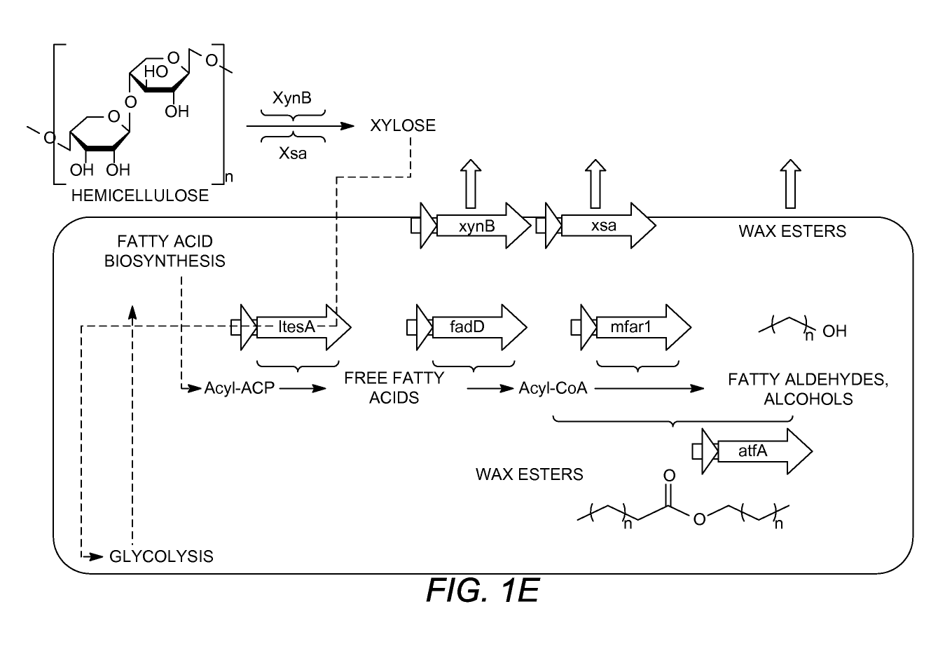

Fatty acid biosynthesis in E. coli, shown in FIG. 1, is negatively regulated by classic product inhibition, where the product, a fatty acyl chain bound to an acyl-carrier protein (ACP), inhibits the fatty acid synthase from generating new fatty acids (Jiang and Cronan 1994; Magnuson et al. 1993). Thus the cell never produces more fat than it needs for building membranes and dividing. The fatty acids are then liberated from the ACP by PlsB or PlsC and proceed to form membrane lipids. Although expression of a cytosolic thioesterase was demonstrated to de-regulate fatty acyl-ACP inhibition by cleaving the thioester bond and producing holo-ACP and free fatty acids, the demonstrated titers were extremely low (ng/L) (Jiang and Cronan 1994).

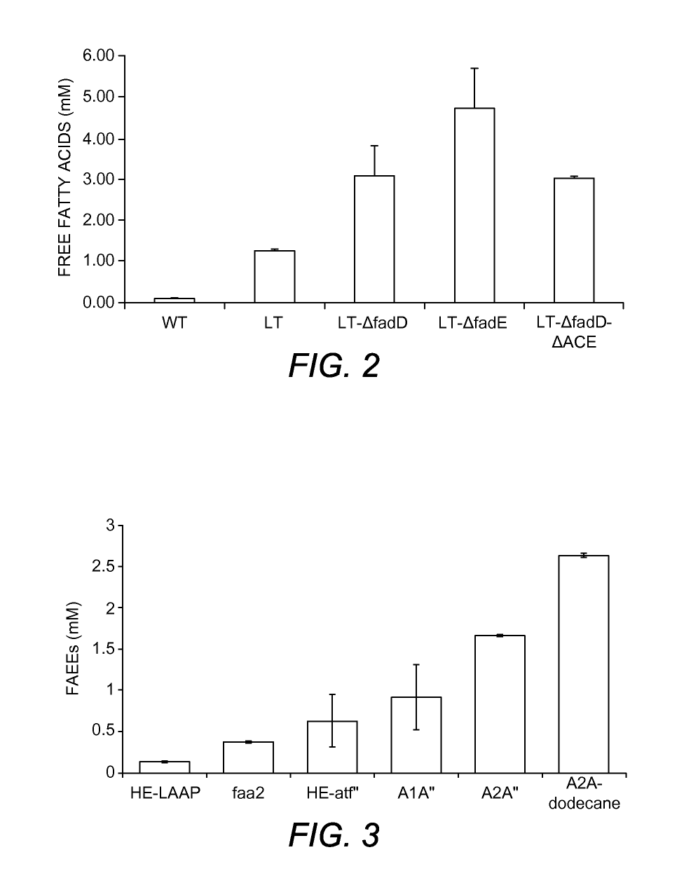

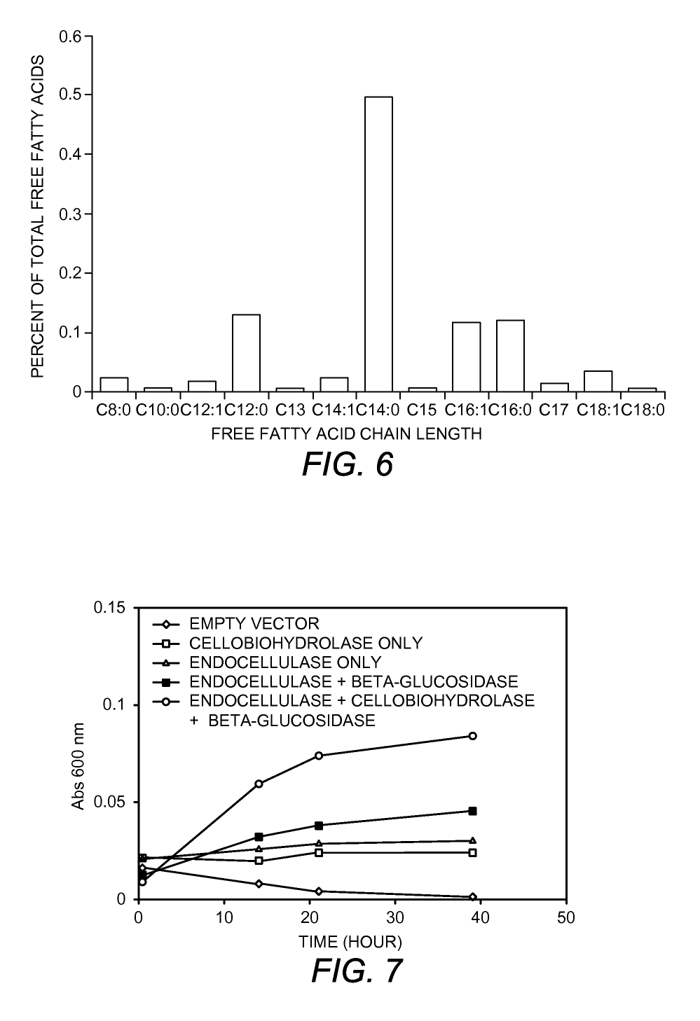

A 10¹²-fold to 500-fold increase compared to previous levels of free fatty acid production was achieved by cytosolic expression of ltesA, a native E. coli thioesterase that is normally found in the periplasm (FIG. 2). LtesA is most specific to C14 fatty acyl-ACPs, although a range of free fatty acids (C8 to C18) was detected (FIG. 6).

In order to further increase efficient production of free fatty acids, competing pathways associated with β-oxidation were eliminated. The first two enzymatic steps for fatty acid degradation require FadD and FadE; thus, these two genes were knocked out, and ltesA was expressed in the cytosol. A dramatic three- to four-fold increase in product titer was achieved, reaching ˜5 mM (FIG. 2). Further attempts at optimization by removal of the acetate-forming reactions (encoded by poxB, pta, and ackA) resulted in free fatty acid production to 3 mM, suggesting that removal of this competing pathway does not greatly aid in over producing fatty acids (FIG. 2). The best strain, LT-ΔfadE, produced approximately 15% of the theoretical limit of fatty acids from 2% glucose (FIG. 2).

### Example 2

### Production of Important Molecules Derived from Fatty Acids

Although fatty acids themselves have great value, they may be modified in order to make other important molecules, including biodiesel equivalents (fatty acid ethyl esters, (FAEEs)), long chain alcohols, and long chain aldehydes, both high-value specialty chemicals that may be used as biofuels.

FAEE Production

Current production of biodiesel is greater than 5 million tons per year and comprises a ˜$4B market (REN21, 2008). Previously, it was shown that E. coli could produce a biodiesel equivalent by esterifying exogenously-added fatty acids with endogenously-produced ethanol (Kalscheuer et al. 2006), a process that would not be economically viable due to the high cost of fatty acids. Having already demonstrated high production levels of fatty acids to 5 mM, a strain was constructed that would produce ethanol by expressing pdc and adhB from Zymomonas mobilis, which encode a pyruvate decarboxylase and an alcohol dehydrogenase, respectively. These strains showed ethanol production to ˜10⁸ mM after 24 h, similar to previous findings (Table 1) (Ingram et al. 1987). Combining the relevant genetic modifications of free fatty acid production (ltesA expression), ethanol production (pdc and adhB expression), and ester production (by expression of the wax ester synthase atfA) resulted in production of FAEEs to 0.14 mM (37 mg/L) (strain HE-LAAP; FIG. 3). Since this strain accumulated significant amounts of free fatty acids that were not converted into the desired product (data not shown), it was reasoned that the cell's endogenous acyl-CoA ligase (fadD) capacity was limiting. Overexpression of faa2, an acyl-CoA ligase from S. cerevisiae, resulted in an approximately 2.5-fold increase in FAEE production to 0.37 mM (96 mg/L) (strain HE-LAAP-faa2; FIG. 3). Another 2-fold increase to 0.63 mM (161 mg/L) was achieved by overexpression of a mutant fadD (harboring two mutations F61L, M335I; FIG. 3). Repairing one mutation in fadD increased production 50% to 0.91 mM (FIG. 3). Expression of an additional copy of atfA resulted in production of 1.7 mM (427 mg/L) FAEEs (strain HE-LAAP-fadDm2-atfA; FIG. 3), which is 13% of the theoretical yield.

TABLE 1
Ethanol production from various E. coli strains

| | DE | DPE | DH | DP | |
|---|---|---|---|---|---|
| Strain | EtOH | EtOH | EtOH | EtOH | DP |
| EtOH (mM) | 115 | 114 | 115 | 119 | ND |

Fatty Acid Ester Production

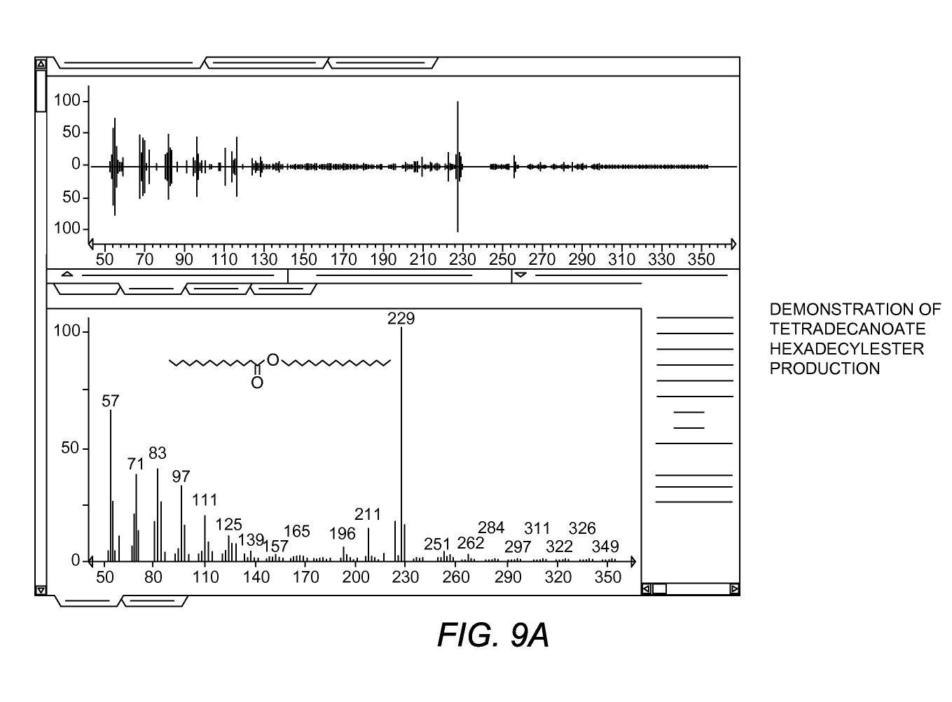

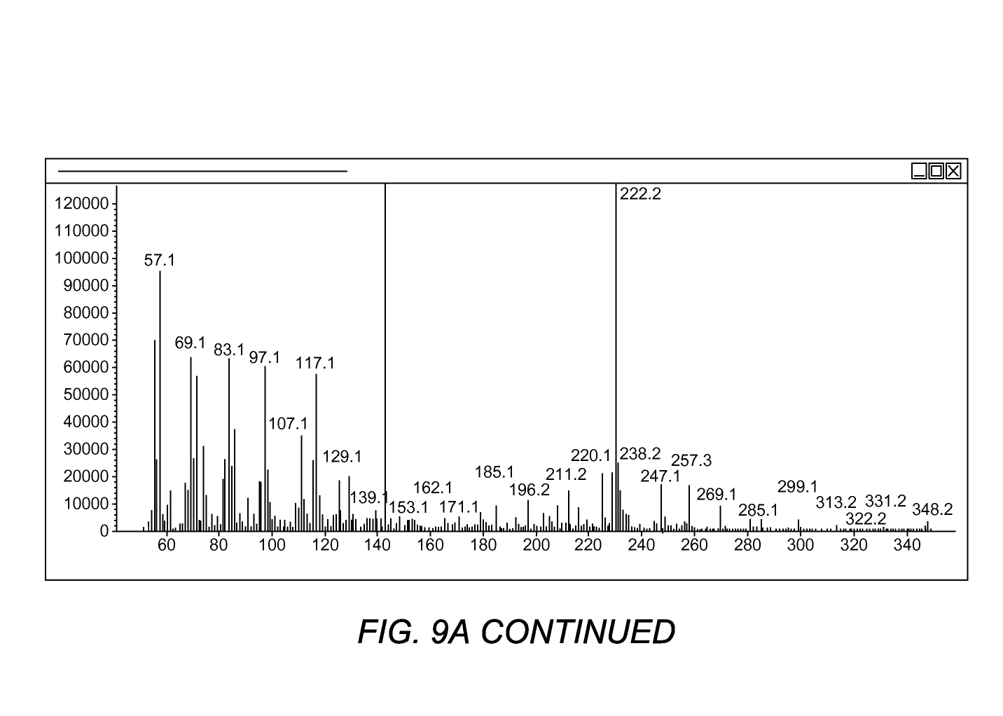

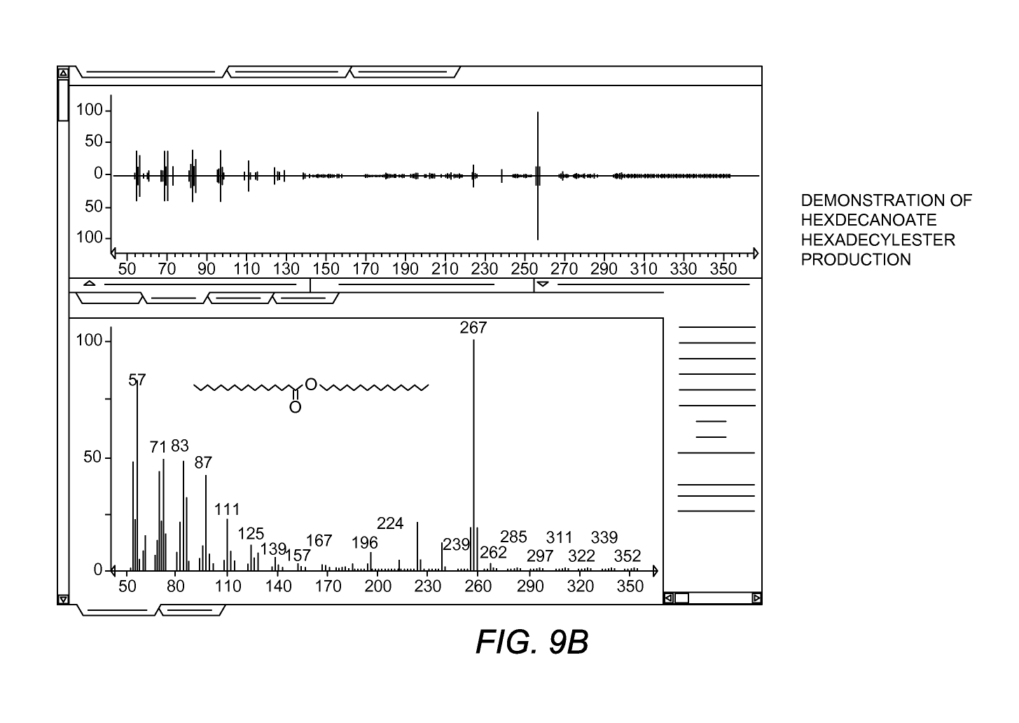

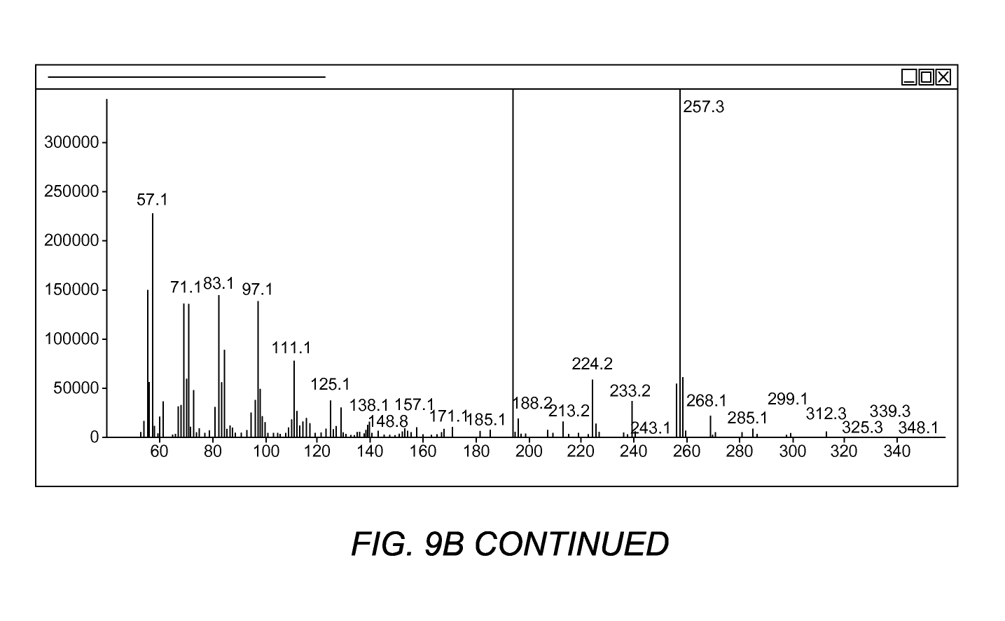

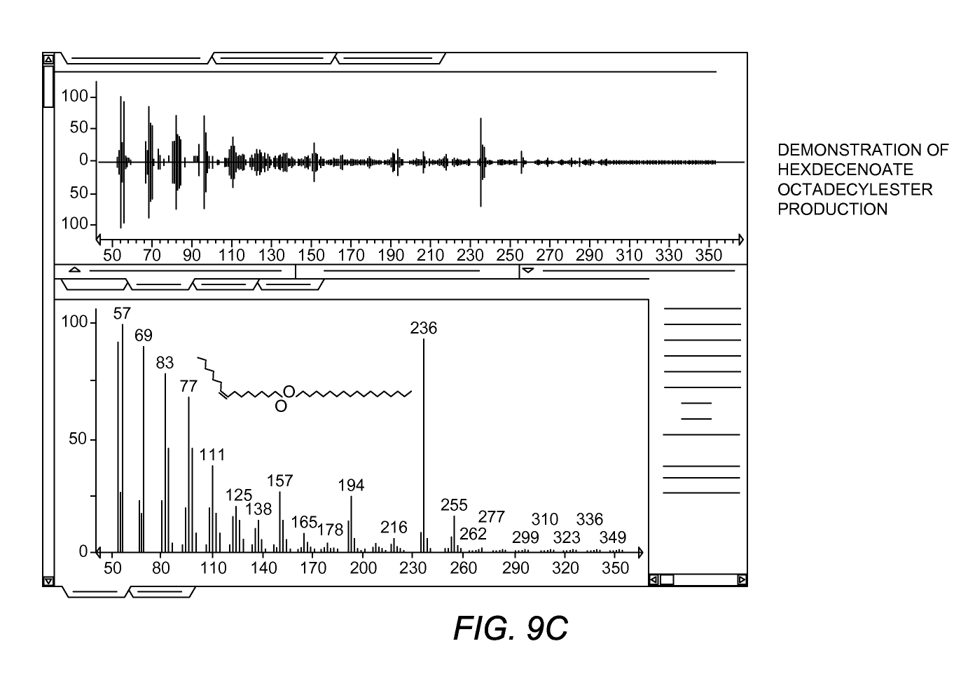

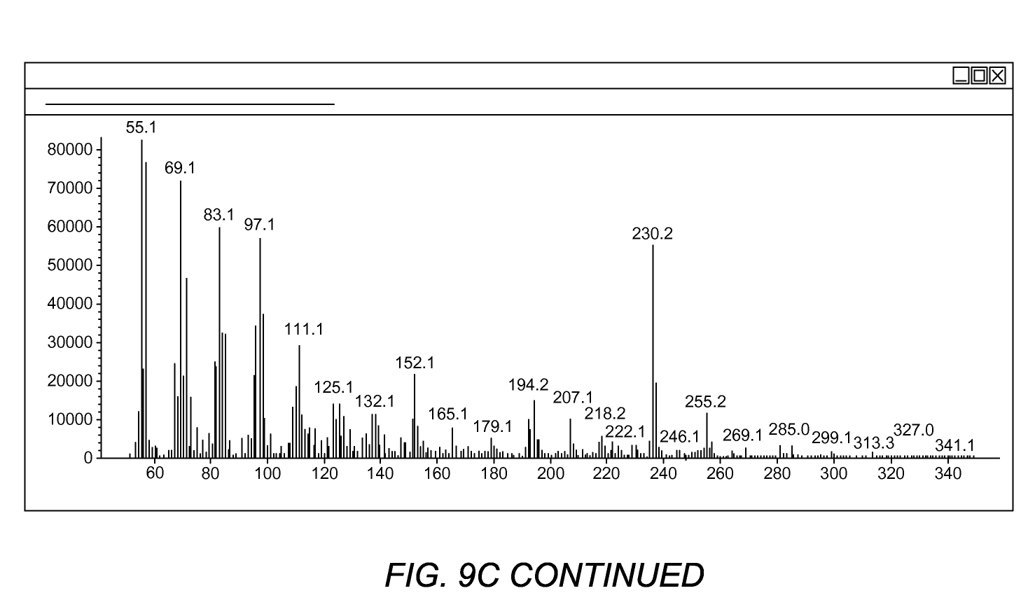

Fatty acid esters (FAEs) or wax esters were produced in a similar fashion to production of FAEEs as described above. ltesA and fadD were overexpressed, and exogenous atfA was expressed. However, no exogenous genes from an ethanol-producing pathway were used. Instead, mfar1 was expressed to produce longer chain alcohols. These longer chain alcohols were then available to AtfA as a substrate for producing wax esters (FIG. 1E). Tetradecanoate hexadecylester, hexdecanoate hexadecylester, and hexdecanoate octadecylester were produced (FIG. 9).

Fatty Alcohol and Aldehyde Production

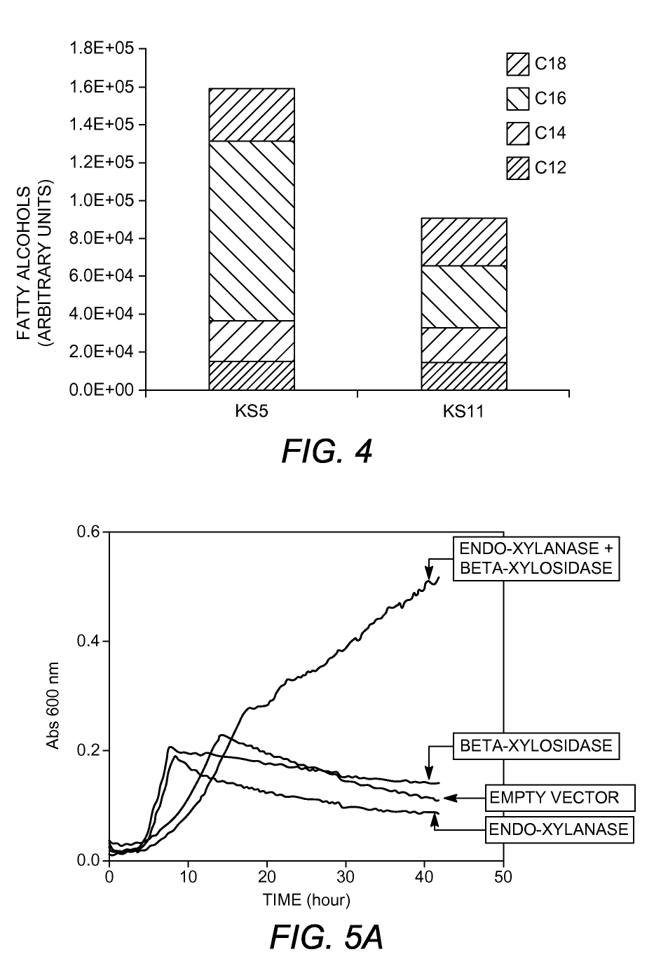

There is a large market for fatty alcohols and aldehydes, which are used predominantly in soaps, detergents, cosmetic additives, pheromones, and flavoring compounds, and potentially as biofuels; their value was approximately $1500/ton (2004 ICIS pricing), with approximately 2 MT produced per year, creating a $3B market (Ahmad 2006). Fatty alcohols are produced either through hydrogenation of fatty acids or FAMEs or through synthesis from petrochemical precursors (Ahmad 2007); both processes require extreme reaction conditions and do not adhere to the principles of green chemistry. Previous identification and expression of fatty alcohol-forming fatty acyl-CoA reductases from plant and mammalian sources has been described (Metz et al. 2000; Cheng and Russell 2004). Here fatty alcohol production ranging from C12 to C18 n-alcohols by engineered E. coli strains expressing either mFar1 (KS5) or acr1 (KS11) in fadE knockout strains was demonstrated (FIGS. 1C and 4).

Fatty aldehyde production is sought after because they are the precursors to alkanes and alkenes, the most energy dense fuels. The biosynthetic pathway for production of alkanes/alkenes requires a decarbonylase that has been partially purified and removes the terminal carbonyl group from fatty aldehydes (Wang and Kolattukudy 1995; Dennis and Kolattukudy 1991). Fatty aldehydes are produced by expressing acr1 (KS11) in fadE knockout strains in which ltesA and fadD are being overexpressed (FIG. 1D). In order to prevent endogenous E. coli alcohol dehydrogenases from converting the fatty aldehydes into fatty alcohols, it is also necessary to knock out or reduce expression of these endogenous dehydrogenase genes or express a decarbonylase in order to compete with the reduction to the alcohol. An E. coli knockout library in the acr1; ΔfadE strain background will be screened to identify genes whose deletion allows for the production of fatty aldehydes.

### Example 3

### Consolidated Bioprocessing: Biomass Polymer Utilization for Biodiesel Production

Although production of second-generation biofuels like FAEEs from sugar has many advantages over ethanol production from sugar, sourcing that sugar from the large available biomass reserves offers an even greater advancement. Unfortunately, sourcing sugar from cellulosic biomass requires the use of costly enzymes to liberate the sugars from pretreated cellulose and hemicellulose. Consolidated bioprocessing, in which the biofuel-producing organism produces glycosyl hydrolases, eliminates the need to add these expensive enzymes and thus reduces costs (Lynd et al. 2005).

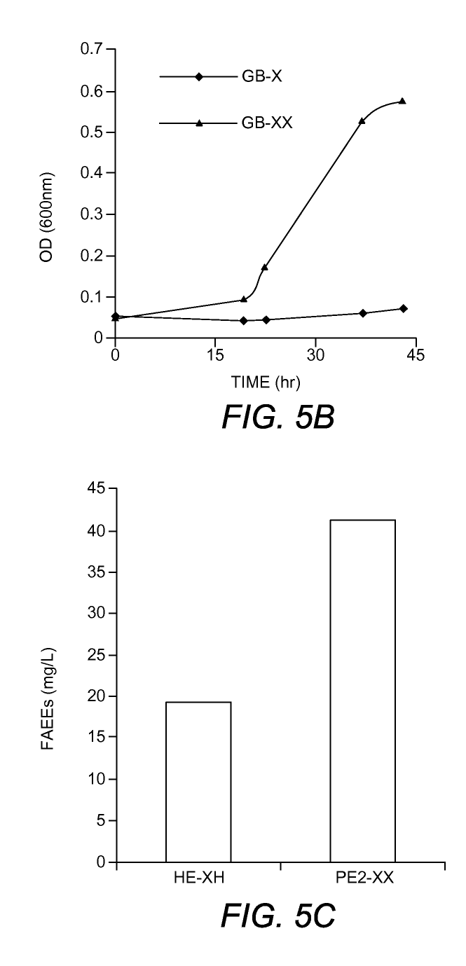

A consolidated bioprocess was achieved by expressing genes encoding an endoxylanase catalytic domain (Xyn10B) from C. stercorarium and a xylanase (Xsa) from Bacteroides ovatus (Adelsberger et al. 2004; Whitehead and Hespell 1990) in E. coli. The hemicellulases were secreted by fusion to the OsmY protein in order to hydrolyze the hemicellulose into xylose, which is catabolized by the native E. coli metabolic pathways (Qian et al.). It may not be necessary, however, for both enzymes to be fused to OsmY. Growth of E. coli transformed with genes encoding the xylan-degrading enzymes individually or at the same time on xylan was demonstrated (FIGS. 5A and B). Expression of these genes with the biodiesel genes resulted in production of FAEEs (FIG. 5C). This consolidated bioprocessing scheme could also be used to produce FAEs from xylan or other biomass polymers.

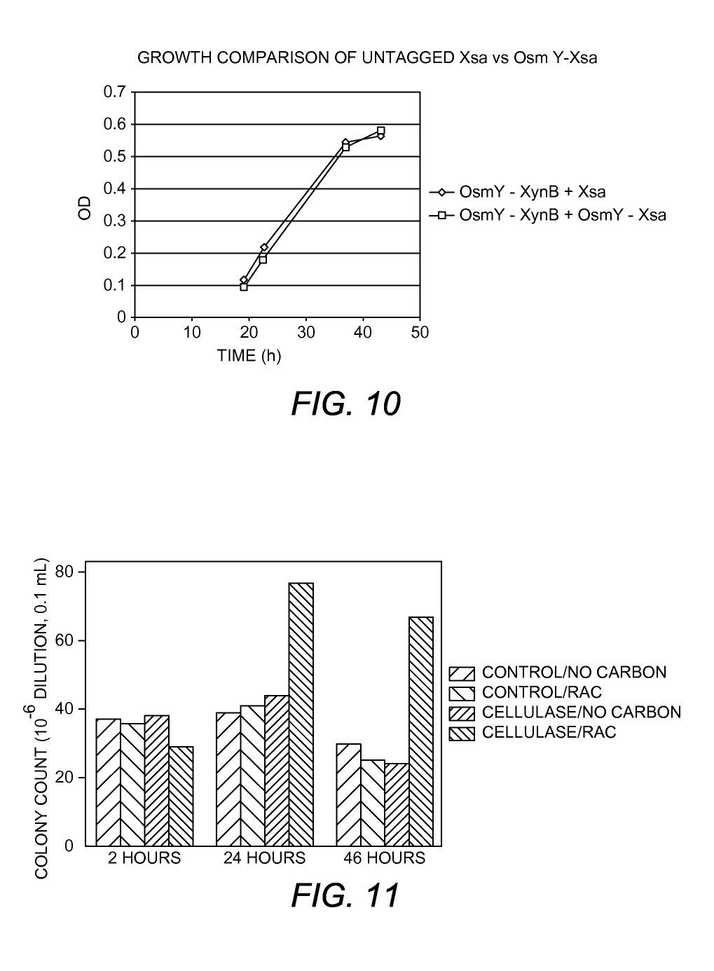

The OsmY tag on the Xsa protein was found to be dispensable for growth on xylo-oligosaccharides. Two plasmids, both containing the OsmY-XynB gene fusion, followed by either an OsmY-Xsa gene fusion, or unfused Xsa, were transformed into BL21 cells. Genes were under control of a propionate promoter. The cells were grown overnight in LB culture supplemented with 200 ug/mL carbenicillin to saturation. The next day, 5 mL of a fresh culture of LB was inoculated with 50 uL of the overnight growth and grown at 37° C. During exponential growth phase (OD of 0.3-0.8), cultures were induced with the addition of sodium propionate to 10 mM for 1-2.5 hours before inoculation into 5 mL of M9 media with 0.2% xylan as the sole carbon source (and 200 ug/mL carbenicillin) and incubation with shaking at 37° C. Growth was determined by monitoring the scattering of the culture at 600 nm (FIG. 10). Growth of these recombinant E. coli will be shown on the hemicellulosic fraction of ionic liquid-treated switchgrass.

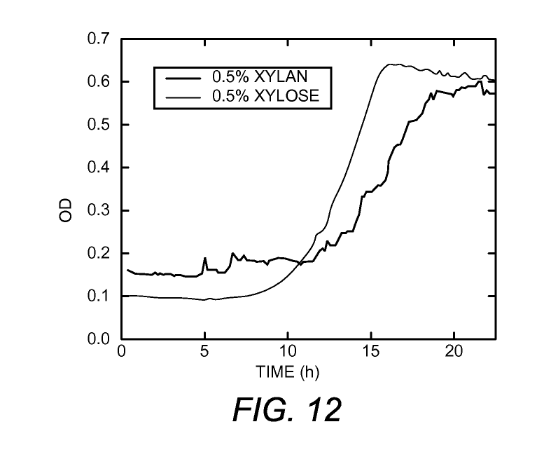

Improved growth on xylan was demonstrated by expressing a different enzyme in place of Xsa. Recombinant E. coli strain MG1655 containing a plasmid bearing XynB, from C. stercorarium, fused to the E. coli gene OsmY and under the control of the E. coli cspD promoter, and the gene encoding the xylobiosidase Gly43F from Cellvibrio japonicus, under the control of the E. coli cstA promoter, were grown in LB medium at 37° C. for 13 hours. 800 uL of MOPS-M9 minimal medium containing either 0.5% beechwood xylan or 0.5% xylose as a sole carbon source was inoculated with 20 uL of the 13 hour growth culture and incubated in a TECAN plate reader at 37° C. Cellular growth was observed by an increase in OD (FIG. 12). Growth of the recombinant E. coli in xylan was almost as fast as growth in xylose. No growth was observed in xylan media in cells lacking either the OsmY-XynB gene or the Gly43F gene.

Growth of E. coli on cellulose was demonstrated. For cellulose utilization, E. coli were transformed with a plasmid containing two enzymes from Cellvibrio japonicus, Cel3B (beta-glucosidase) expressed without being fused to OsmY and the catalytic domain of Cel6A (cellobiohydrolase) fused to OsmY (J Bact, vol 190, p. 5455), as well as a codon-optimized version of the catalytic domain of a cellulase from Bacillus subtilis D04 (J Biol Chem, vol 270, p. 26012). All genes were under control of the lacUV5 promoter. E. coli were grown and induced in LB media before transferring ( 1/100) to M9 media containing 0.2% carboxymethyl-cellulose (FIG. 7).

In addition, E. coli expressing a cellulase and a beta-glucosidase were demonstrated to grow on phosphoric swollen cellulose (PASC). A plasmid was constructed bearing the following: the beta-glucosidase gene cel3A, from Cellvibrio japonicus, under the control of the promoter for the wrbA gene as found in the E. coli MG1655 genome; a codon-optimized version of the glycoside hydrolase catalytic domain found in the cel gene from Bacillus subtilis sp. D04, fused on its N-terminus with the OsmY protein from E. coli, under the control of the promoter for the cspD gene as found in the E. coli MG1655 genome; a low-copy origin of replication (SC101**); and the ampicillin resistance gene bla. The plasmid was transformed into BL21 cells, and the cells were grown in LB medium supplemented with 100 ug/mL carbenicillin for approximately 18 hours at 37° C.

MOPS-M9 medium (7 ml) with 100 ug/mL carbenicillin and with either no source of carbon or with 0.5% regenerated amorphous cellulose (RAC) (prepared as described in Metabolic Engineering, vol 9, p. 87, 2007), was inoculated with 1 mL of the overnight growth bearing the plasmid described above, or a control plasmid lacking cellulase or beta-glucosidase genes and carrying only antibiotic resistance. Cultures were incubated at 37° C. with shaking.

At intervals, samples of the cultures were taken and diluted in LB medium to 10-6 concentration. 100 uL of this dilution were plated on LB-agar plates, without antibiotics, and the plates were incubated overnight at 37° C. Colonies were counted. Significant (˜2×) growth was seen in the cellulase-producing strain in the presence of cellulose, while little or no growth was seen in the absence of either cellulase production or carbon source (FIG. 11). Growth of these recombinant E. coli will be shown on the cellulosic fraction of ionic liquid-treated switchgrass.

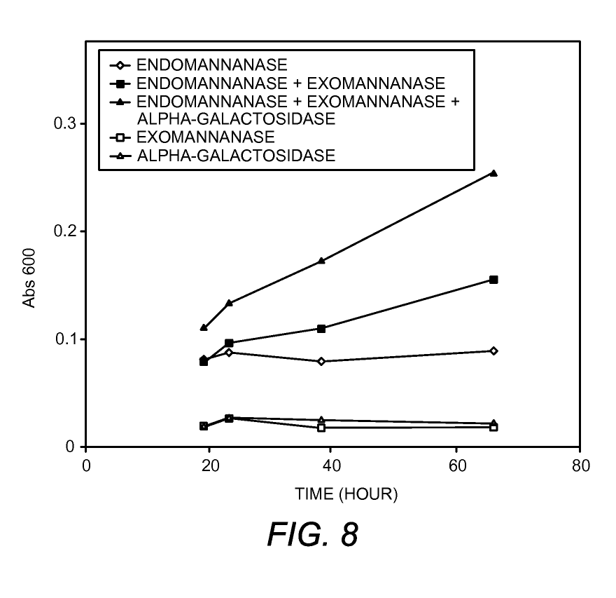

Growth of E. coli on mannan was demonstrated. For mannan utilization, three enzymes from Cellvibrio japonicus, the catalytic domains of Man26A (endomannanase), Man5D (exomannase), and Aga27A (alpha-galactosidase, a debranching enzyme) were used (J Bact, vol 190, p. 5455). All catalytic domains were fused to the OsmY protein. Catalytic domains were individually expressed in a co-culture of multiple organisms acting together to degrade the locust bean gum into mannose and galactose (FIG. 8). Co-cultures of E. coli secreting individual enzymes were grown in M9 media containing 0.2% locust bean gum (galactomannan). E. coli may be transformed with all three catalytic domains on one plasmid.

E. coli may be engineered to express enzymes for both hemicellulose and cellulose degradation: an OsmY-XynB gene fusion, followed by either an OsmY-Xsa gene fusion, or unfused Xsa, as well as Cel3B (beta-glucosidase) without being fused to OsmY, and the catalytic domain of Cel6A (cellobiohydrolase) fused to OsmY. These E. coli will be shown to utilize simultaneously both the cellulosic and hemicellulosic fractions of ionic liquid-treated switchgrass.

These E. coli engineered to utilize cellulose and mannan can be further manipulated to produce fatty acid ethyl esters, fatty alcohols, fatty aldehydes, and other fatty acid-derived compounds as described in Example 2 for direct conversion of cellulose and mannan into these valuable products. Furthermore, combining the xylan, cellulose, and mannan degradation pathways in one organism will allow for one cell to use whole biomass as a carbon source.

Here, the importance and utility of the fatty acid biosynthesis pathway was demonstrated for production of a class of important chemicals and biofuels by E. coli in a consolidated bioprocess utilizing hemicellulose as a feedstock. The high titers will enable transition to industrial processes for production of biofuels or chemicals. Importantly, these fatty acid-derived molecules are not toxic to the cell, which is a problem with the less-energy dense, lower alcohols that have been targeted as important, next-generation biofuels but suffer from low titers (Atsumi et al. 2008; Steen et al. 2008). In addition a demonstration of high production levels of a biofuel, the production of biofuel from hemicellulose was shown, which was an imperative, yet unrealized goal of the field. The strategy of deregulating fatty acid biosynthesis, identifying the key rate limiting steps for production of fatty acid derived biofuels, and producing these biofuels from inexpensive, renewable, plant-derived biomass in a consolidated bioprocess opens the field of metabolic engineering for the production of highly energy dense, second-generation biofuels and related chemicals from renewable resources in a wide range of organisms.

### Example 4

### Materials and Methods

Reagents

All chemicals were purchased from Sigma-Aldrich (St. Louis, Mo.) and include fatty acid methyl ester standards, fatty acid ethyl ester standards, fatty aldehyde standards, and fatty alcohol standards.

Strains and Plasmids

E. coli DH1 was utilized as the wild-type strain for all studies. Knockouts of fadD, fadE, pta, poxB, and ackA, were performed as previously described (Datsenko and Wanner 2000). E. coli DH10B and DH5α were used for bacterial transformation and plasmid amplification in the construction of the expression plasmids used in this study; E. coli fadDKO was utilized to overexpress fadD. Native E. coli genes were cloned from DH1. mFAR1 (Mus musculus, GenBank Accession BC007178) was synthesized and codon optimized for E. coli expression (Epoch biolabs). atfA (Acinetobacter sp. strain ADP1) was synthesized (Epoch biolabs) (Cheng and Russell 2004). acr1 (Acinetobacter baylyi) was kindly provided by Chris Somerville (University of California, Berkeley). pdc and adhB were cloned from Z. mobilis genomic DNA (ATCC 31821). FAA2 was cloned from Saccharomyces cerevisiae (BY4742) genomic DNA. Plasmids were constructed using the “Sequence and Ligation Independent Cloning” (SLIC) method (Li and Elledge 2007). All genes were over-expressed under the control of the IPTG-inducible lacUV5 or trc promoters as indicated. For strain and plasmid construction, strains were cultivated at 37° C. in Luria-Bertani medium with the appropriate antibiotics (50 μg/L ampicillin (Amp), 20 μg/L chloramphenicol (Cam), 5 μg/L tetracycline (Tet)). To characterize production levels of fatty acid-derived molecules, strains were grown in M9 minimal medium with the appropriate antibiotic and induced at an optical density measured at a wavelength of 600 nm (OD600) of 0.5-1 with 500 μM IPTG.

TABLE 2

| | Replication | Overexpressed | | |
|---|---|---|---|---|
| Plasmids | Origin | Genes | Resistance | Reference |
| pACYC184 | p15a | | | {Chang, |
| | | | | 1978 |
| | | | | #119} |
| pBBR1MCS-3 | pBBR | | Tet | 27 |
| pBR322 | pBR322 | | Amp | 28 |
| pKS01 | p15a | placUV5: LtesA | Cam | This study |
| pKS13 | pBBR | placUV5: pdc, | Tet | This study |
| | | adhB | | |
| pKS17 | pBBR | placUV5: pdc, | Tet | This study |
| | | adhB, atfA | | |
| pKS18 | pBBR | placUV5: mFar1 | Tet | This study |
| pKS19 | pBBR | placUV5: acr1 | Tet | This study |
| pKS100 | pBR322 | pTRC: fadD | Amp | This study |
| pKS101 | pBR322 | pTRC: FAA2 | Amp | This study |
| | pBR322 | pTRC: fadD, | Amp | This study |
| | | atfA | | |
| | p15a | placUV5: LtesA, | Cam | This study |
| | | atfA | | |
| | pBR322 | pTRC: FAA2, | Amp | This study |
| | | atfA | | |
| | p15a | placUV5: LtesA, | Cam | This study |
| | | mfar1 | | |
| | p15a | placUV5: LtesA, | Cam | This study |
| | | acr1 | | |
| pKS5 | p15a | placUV5: mFar1 | Cam | This study |
| pKS11 | p15a | placUV5: acr1 | Cam | This study |
| pGB1 | pBR322 | pPro: xyn10B, | Amp | This study |
| | | xsa | | |
| pGB2 | pBR322 | placUV5: fadD, | Amp | This study |
| | | xyn10B, xsa | | |

Metabolite Analysis

Total free fatty acids were extracted from 5 mL cultures by addition of 500 μL HCl and 5 mL of ethyl acetate, spiked with 10 mg/L of methyl nonadecanoate as an internal standard. The culture tubes were vortexed for 15 seconds followed by shaking at 200 rpm for 20 minutes. The organic layer was separated, and a second extraction was performed by addition of another 5 mL ethyl acetate to the culture tubes. The free fatty acids were then converted to methyl esters by addition of 200 μL TMS-diazomethane, 10 μL HCl, and 90 μL MeOH (Aldai et al. 2005). This reaction was allowed to proceed for 2 hr and then was applied to a Thermo Trace Ultra gas chromatograph (GC) equipped with a Triplus AS autosampler and a TR-WAXMS column (Thermo Scientific). The GC program was as follows: initial temperature of 40° C. for 1.2 min, ramped to 220° C. at 30° C./min and held for 3 min. Final quantification analysis was performed with Xcalibur software.

Fatty acid ethyl esters (FAEEs), fatty alcohols, and fatty aldehydes were extracted from cultures by addition of 10% (v/v) ethyl acetate, spiked with 10 mg/L methyl nonadecanoate, followed by shaking at 200 rpm for 20 min. Analysis of FAEEs was performed on an HP 6890 Series GC with an Agilent 5973 Network MSD equipped with a DB5 column (Thermo). The GC program was the same as for quantifying FAMES. Fatty alcohols and aldehydes were separated with a TR-Wax column (Agilent). The GC program was as follows: initial temperature of 70° C., held for 1 min, ramped to 240° C. at 25° C./min and held for 3 min.

Ethanol was measured by sampling 1 mL of culture, centrifuging 14 k rpm, 5 min, and applying the supernatant to an Agilent 1100 series HPLC equipped with an Aminex HPX-87H ion exchange column (Biorad). The solvent (4 mM H₂SO₄) flow rate was 0.6 mL/min, and the column was maintained at 50° C. All metabolites were detected with an Agilent 1200 series DAD and RID detectors.

### REFERENCES

Adelsberger, H., Hertel, C., Glawischnig, E., Zverlov, V. V. & Schwarz, W. H. Enzyme system of Clostridium stercorarium for hydrolysis of arabinoxylan: reconstitution of the in vivo system from recombinant enzymes. Microbiology 150, 2257-66 (2004).
Ahmad, W. R. a. S. The changing world of oleochemicals. Palm Oil Developments, 15-28 (2006).
Ahmad, W. R. a. S. Palm oil and palm kernel oil as raw materials for basic oleochemicals and biodiesel. Eur J Lipid Sci Technol 109, 433-439 (2007).
Aldai, Noelia, B. E. M. A. I. N. D. J. T. K. O. Derivatization of fatty acids and its application for conjugated linoleic acid studies in ruminant meat lipids. Journal of the Science of Food and Agriculture 85, 1073-1083 (2005).
Atsumi, S., Hanai, T. & Liao, J. C. Non-fermentative pathways for synthesis of branched-chain higher alcohols as biofuels. Nature 451, 86-9 (2008).
Bligh, E. G. a. D., W. J. A rapid method of total lipid extraction and purification. Can. J. Biochem. Physiol. 19, 156-159 (1959).
Bolivar, F. Construction and characterization of new cloning vehicles. III. Derivatives of plasmid pBR322 carrying unique Eco RI sites for selection of Eco RI generated recombinant DNA molecules. Gene 4, 121-36 (1978).
Brown A C, K. B. Hydrocarbon content and its relationship to physiological state in the green alga Botryococcus braunii. Phytochemistry 8, 543-547 (1969).
Cheng, J. B. & Russell, D. W. Mammalian wax biosynthesis. I. Identification of two fatty acyl-Coenzyme A reductases with different substrate specificities and tissue distributions. J Biol Chem 279, 37789-97 (2004).
Datsenko, K. A. & Wanner, B. L. One-step inactivation of chromosomal genes in Escherichia coli K-12 using PCR products. Proc Natl Acad Sci USA 97, 6640-5 (2000).
Dehesh, K., Jones, A., Knutzon, D. S. & Voelker, T. A. Production of high levels of 8:0 and 10:0 fatty acids in transgenic canola by overexpression of Ch FatB2, a thioesterase cDNA from Cuphea hookeriana. Plant J 9, 167-72 (1996).
Dennis, M. W. & Kolattukudy, P. E. Alkane biosynthesis by decarbonylation of aldehyde catalyzed by a microsomal preparation from Botryococcus braunii. Arch Biochem Biophys 287, 268-75 (1991).
Fischer, C. R., Klein-Marcuschamer, D. & Stephanopoulos, G. Selection and optimization of microbial hosts for biofuels production. Metab Eng 10, 295-304 (2008).
Hall, M. J. & Ratledge, C. Lipid Accumulation in an Oleaginous Yeast (Candida 107) Growing on Glucose Under Various Conditions in a One- and Two-Stage Continuous Culture. Appl Environ Microbiol 33, 577-584 (1977).
Hill, J., Nelson, E., Tilman, D., Polasky, S. & Tiffany, D. Environmental, economic, and energetic costs and benefits of biodiesel and ethanol biofuels. Proc Natl Acad Sci USA 103, 11206-10 (2006).
Ingram, L. O., Conway, T., Clark, D. P., Sewell, G. W. & Preston, J. F. Genetic engineering of ethanol production in Escherichia coli. Appl Environ Microbiol 53, 2420-5 (1987).
Jiang, P. & Cronan, J. E., Jr. Inhibition of fatty acid synthesis in Escherichia coli in the absence of phospholipid synthesis and release of inhibition by thioesterase action. J Bacteriol 176, 2814-21 (1994).
Kalscheuer, R., Stolting, T. & Steinbuchel, A. Microdiesel: Escherichia coli engineered for fuel production. Microbiology 12, 2529-36 (2006).
Knights B A, B. A., Conway E, Middleditch B S. Hydrocarbons from the green form of the freshwater alga Botryococcus braunii. Phytochemistry 9, 1317-1324 (1970).
Kovach, M. E., Phillips, R. W., Elzer, P. H., Roop, R. M., 2nd & Peterson, K. M. pBBR1MCS: a broad-host-range cloning vector. Biotechniques 16, 800-2 (1994).
Li, M. Z. & Elledge, S. J. Harnessing homologous recombination in vitro to generate recombinant DNA via SLIC. Nat Methods 4, 251-6 (2007).
Lu, X., Vora, H. & Khosla, C. Overproduction of free fatty acids in E. coli: implications for biodiesel production. Metab Eng 10, 333-9 (2008).
Lynd, L. R., van Zyl, W. H., McBride, J. E. & Laser, M. Consolidated bioprocessing of cellulosic biomass: an update. Curr Opin Biotechnol 16, 577-83 (2005).
Magnuson, K., Jackowski, S., Rock, C. O. & Cronan, J. E., Jr. Regulation of fatty acid biosynthesis in Escherichia coli. Microbiol Rev 57, 522-42 (1993).
Metz, J. G., Pollard, M. R., Anderson, L., Hayes, T. R. & Lassner, M. W. Purification of a jojoba embryo fatty acyl-coenzyme A reductase and expression of its cDNA in high erucic acid rapeseed. Plant Physiol 122, 635-44 (2000).
Qian, Z. G., Xia, X. X., Choi, J. H. & Lee, S. Y. Proteome-based identification of fusion partner for high-level extracellular production of recombinant proteins in Escherichia coli. Biotechnol Bioeng 101, 587-601 (2008).
Steen, E. J. et al. Metabolic engineering of Saccharomyces cerevisiae for the production of n-butanol. Microb Cell Fact 7, 36 (2008).
Wang, X. & Kolattukudy, P. E. Solubilization and purification of aldehyde-generating fatty acyl-CoA reductase from green alga Botryococcus braunii. FEBS Lett 370, 15-8 (1995).
Whitehead, T. R. & Hespell, R. B. The genes for three xylan-degrading activities from Bacteroides ovatus are clustered in a 3.8-kilobase region. J Bacteriol 172, 2408-12 (1990).
REN21. Renewables 2007 global status report. Worldwatch Institute, Washington D.C. (2008).

What is claimed is:

1. A method for producing biodiesel equivalents and other fatty acid derivatives from a biomass polymer, comprising:
(a) providing a genetically modified host cell, wherein the host cell comprises one or more recombinant nucleic acids selected from the group consisting of:
i. one or more recombinant nucleic acids encoding a thioesterase, a fatty acyl-coA synthetase, an acyl-transferase, an alcohol dehydrogenase, a pyruvate decarboxylase, and one or more biomass polymer-degrading enzymes;
ii. one or more recombinant nucleic acids encoding a thioesterase, a fatty acyl-coA synthetase, a fatty alcohol-forming fatty acyl-coA reductase, and one or more biomass polymer-degrading enzymes; and
iii. one or more recombinant nucleic acids encoding a thioesterase, a fatty acyl-coA synthetase, a fatty acyl-coA reductase, and one or more biomass polymer-degrading enzymes,
wherein the one or more biomass polymer-degrading enzymes are secreted from the genetically modified host cell;
(b) culturing the host cell in a medium to form a culture such that the one or more recombinant nucleic acids are expressed in the cell, wherein the medium comprises a biomass polymer as a carbon source for the host cell; and
(c) optionally extracting biodiesel equivalents and other fatty acid derivatives from the culture medium.

2. The method of claim 1, wherein the biodiesel equivalents and other fatty acid derivatives are fatty alcohols.

3. The method of claim 1, wherein the biodiesel equivalents and other fatty acid derivatives are fatty aldehydes.

4. The method of claim 1, wherein the acyl-transferase is a wax ester synthase.

5. The method of claim 1, wherein the fatty acyl-coA synthetase is fadD.

6. The method of claim 1, wherein the host cell is modified such that expression of an endogenous fatty acyl-coA dehydrogenase is attenuated relative to the level of expression in a non-modified cell.

7. The method of claim 1, wherein the host cell is a bacterial cell.

8. The method of claim 7, wherein the bacterial cell is an E. coli cell.

9. The method of claim 1, wherein the biomass polymer is hemicellulose or cellulose.

10. The method of claim 9, wherein the hemicellulose is xylan and the one or more biomass polymer-degrading enzymes are a xylanase and a protein comprising an endoxylanase catalytic domain.

11. The method of claim 1, wherein the one or more biomass polymer-degrading enzymes are a protein containing a cellobiohydrolase catalytic domain, a beta-glucosidase, and a protein containing a cellulase catalytic domain.

12. A genetically modified host cell, comprising
one or more recombinant nucleic acids selected from the group consisting of:
i. one or more recombinant nucleic acids encoding a thioesterase, a fatty acyl-coA synthetase, an acyl-transferase, an alcohol dehydrogenase, a pyruvate decarboxylase, and one or more biomass polymer-degrading enzymes;
ii. one or more recombinant nucleic acids encoding a thioesterase, a fatty acyl-coA synthetase, a fatty alcohol-forming fatty acyl-coA reductase, and one or more biomass polymer-degrading enzymes; and
iii. one or more recombinant nucleic acids encoding a thioesterase, a fatty acyl-coA synthetase, a fatty acyl-coA reductase, and one or more biomass polymer-degrading enzymes,
wherein the one or more biomass polymer-degrading enzymes are secretory enzymes.

13. The genetically modified host cell of claim 12, wherein the host cell produces fatty alcohols.

14. The genetically modified host cell of claim 12, wherein the host cell produces fatty aldehydes.

15. The genetically modified host cell of claim 12, wherein the acyl-transferase is a wax ester synthase.

16. The genetically modified host cell of claim 12, wherein the acyl-coA synthetase is fadD.

17. The genetically modified host cell of claim 12, wherein the host cell is modified such that expression of an endogenous fatty acyl-coA dehydrogenase is attenuated relative to the level of expression in a non-modified cell.

18. The genetically modified host cell of claim 12, wherein the host cell is a bacterial cell.

19. The genetically modified host cell of claim 18, wherein the bacterial cell is an E. coli cell.

20. The genetically modified host cell of claim 12, wherein the one or more biomass polymer-degrading enzymes are a xylanase and a protein comprising an endoxylanase catalytic domain.
